SEQUENCE LISTING (34 sequences)

SEQ ID NO: 1 - DNA, 2880 bp, Vibrio sp.
atgacgggctctagcagagagactttggccatggagatgaaccagtccgcagcctccgtgtccttcggcaagaggaacggctgctacgtgaacaggagcgtggccgtgatcctggggctcttgttcgtcagcgccacagtggccacggggcttctcgtgtattactacgcgccgcaggtcagagaggcgggagaccctctcaacctcgccagaacctcgctagccacggaacgccccatggtccctaccacccccatgaccccagcgtcagaggtcaaacccaaaatcgacgtgaggcttccgcgatccgtgaaacccttgcactacaaggtcaaactccagcctctcatcaacgggaacttcagcatcctgggttacgtcgaggtcgaggtcgaggtgctggaggcgacgtcgaatgtcaccctccacatcgctgacattgtgaccaagaatgagacgataaagctcgccccatccgaccaagtccaagggcctggcatcggcatcaacaaacacagctatgacaacgagagacagttctacgtcgccgaactcggagaagaactcgaggtcgggaagaaatacgtcttgtcgatggacttcgaagggtacctcaacgaccagctccacggcttctacagtcgacttacaaggcgaggacggagcgacaggttgatcgcctcgacccagttccagcccacggacgcccgcagagctttcccttgcttcgacgagcccgggatgaaggcgactttcgaggtgtacctgggcaaggaggagggcatgtcgtccatttcgaacatgcccaagtttgaatccatccccatagagggacagccaggatgggtgtgggaccactttaacacaagcgtccccatgtccacttacttggttgccttcgtcatctcggatttctcccacatgaactccaccgccaatgaccacgtcttgttcagagtgtgggcccgcaaggcagccatcgagcaggccaactacgcgctgacaacaggacctgacatcctgaccttcttcgagggctacttcaacgtgcctttcccacttcccaagcaggacatgatcgccatcccggacttctcggcgggagccatggagaactggggcctcatcacctacagagaaacggccagtttgtacgaccccgcagtctccgccgcgtctaacaagcagcgggtggtagtggtagttgcgcacgaactcgcccatcagtggttcggcaatttggtcacgcccgagtggtggacggacctctggctcaacgagggcttcgagtccttcatggagtacctgggtgttgatcattccgagccctcctggaagatgatggagcagttcgtccccgatgacctccacgacgtcttcgccatcgactgcctggaatcctcgcatcctatcagcatccctgtcggccacccggacgagatcaacgagattttcgatcgtatctcctacgctaagggtggcttcgattatccgcatgatgaaccacttcttgacggaggccaccttcaggaagggcctctccaattacctcacggacctgaaatacagaacgcagagcaggacgacctttggcagtacctgacgacagcggcctacgaggacaacacactccccacagacatcagtgttaagaaaatcatggacacttggacactgcagatgggttaccctgtgatcaaggtgacgcgtagcagtgacggcacttcggccacggttaccaagaacgcttcctcctcgtcaagaaccccaactcgacggacacccacgactacaagtggtgggtccctctgtcctacaccacggagaccagcccggacttcgagacgacgaagccccagcggtggatgatggacacggagcaacagctcaccatttcctctctcccggcgaaaggacaagtgggtgatcttcaacgtgcaggagacgggttattacagggtcaactacgacgccgagaactggaacctgatcatccagcaactcaaggaccagcatgagtcgattcacgtcatcaacagagcgcagattatcgacgatgttttgaacctcgcgcgtgccggccaggtatcctacgacacggccctgagcgtgaatgcctacctgggcaaggaagtggagtacgtaccttgggacaccgctctcaacaatctgggttacctggaaaatatgttcactcggtccagtggctacggcgatctcaagagctacctcctggacatcctgatccccctctacaactccgtgggcttcgaggacaacctcaacgacccccacttggaccagtacaagcgggttaaggctctctcttgggcgtgcaacctcggctaccaagactgcgtggataattcgcagtctctcttcaatacgtggtcgacggtctactgcacgggcgtggctgagggcggcgaggaggagtggaacttcgcgtgggaacagtacatcaactcgaacgtcgccacagagaaggccaagctcctgtccgccatgggttgtaccaaggaagtgtggattctgtccaggtacctcgacatggcgttcacggaaggcagcggcatccggaagcaggacgcgtcgcaggtcttcgccgccgtcgccaggaatgacgtggggcgctacctggcctggaactacctcagggaccagtggcagaagatcgccgattattacggatcaggtttctttgccattgcccggatcataaaggccgccacaagagcctttaacacgaagctggagttggcggagctggaactcttcaagcaacaacacgaaggccagctgggcacagcaacccgcgccgtcgaccaagccatcgagagaacagaaaataacattaagtggatggataacaactatgac

SEQ ID NO: 2 - protein, 959 aa, Vibrio sp.
MTGSSRETLAMEMNQSAASVSFGKRNGCYVNRSVAVILGLLFVSATVATGLLVYYYAPQVREAGDPLNLARTSLATERPMVPTTPMTPASEVKPKIDVRLPRSVKPLHYKVKLQPLINGNFSILGYVEVEVEVLEATSNVTLHIADIVTKNETIKLAPSDQVQGPGIGINKHSYDNERQFYVAELGEELEVGKKYVLSMDFEGYLNDQLHGFYSRLTRRGRSDRLIASTQFQPTDARRAFPCFDEPGMKATFEVYLGREEGMSSISNMPKFESIPIEGQPGWVWDHFNTSVPMSTYLVAFVISDFSHMNSTANDHVLFRVWARKAAIEQANYALTTGPDILTFFEGYFNVPFPLPKQDMIAIPDFSAGAMENWGLITYRETAMLYDPAVSAASNKQRVVVVVAHELAHQWFGNLVTPEWWTDLWLNEGFASFMEYLGVDHSEPSWKMMEQFVPDDLHDVFAIDCLESSHPISIPVGHPDEINEIFDRISYAKGASIIRMMNHFLTEATFRKGLSNYLTDLKYQNAEQDDLWQYLTTAAYEDNTLPTDISVKKIMDTWTLQMGYPVIKVTRSSDGTSATVTQERFLLVKNPNSTDTHDYKWWVPLSYTTETSPDFETTKPQRWMMDTEQQLTISSLPAKDKWVIFNVQETGYYRVNYDAENWNLIIQQLKDQHESIHVINRAQIIDVLNLARAGQVSYDTALSVNAYLGKEVEYVPWDTALNNLGYLENMFTRSSGYGDLKSYLLDILIPLYNSVGFEDNLNDPHLDQYKRVKALSWACNLGYQDCVDNSQSLFNTWSTVYCTGVAEGGEEEWNFAWEQYINSNVATEKAKLLSAMGCTKEVWILSRYLDMAFTEGSGIRKQDASQVFAAVARNDVGRYLAWNYLRDQWQKIADYYGSGFFAIARIIKAATRAFNTKLELAELELFKQQHEGQLGTATRAVDQAIERTENNIKWMDNNYD

SEQ ID NO: 3 - DNA, 1309 bp, Vibrio sp.
atgctcagcgccacggcctggttacggggcgcttaggtttttgtatcttccttcttctttcttttattcatctcttcgcttctccttcctcctctccttcttctccatctcactcctctctttatacctcttcctcgttcactcccttccctctctcgactcagtacttcactttccttttcttatccttctcttcctctttcgtcctcctccctccccctcccgtacctcgccctcactcagtcatcctcctcctccactcaccacctccatctaccctccgctctcccccaccttacccccccatctcagccccccataccacccctcaccttaccaccttacatcctcacctcccccttacccccttccatctcatcctcgctctcactcctcacccatcttccatctcatccaccctccatctcaccccgcctctccttccctccactcctccttccatctcaccccccttccccctcccctcaccaccctccatctcacccccctcttcccccccctcacccacctcgctctccccctctcatcctctcccagctcccattcaacccctcacccttcccctcacaccttgccacccccctcttacccccctacccttaccccaccttcctctcactcccccctcgcccatctacctcctcaccctcgtcctccccacttccatctcacccccccccttactttctccctcttccagcaataccgcttccttcacctgctcattccctgtacaactcggtcggcttcgaggacgaccttcaaggaccacatctggaccagtacaagcgcgcgatggctctgaggtggacctgtggcctcggctatgtggactgcgtggatcggtcggtcctccagtttgaggagtggattaataatggaagtgacgtgtccccgaacctgaagtcgacggtgtactgcagtgccatcgctgccggcggcgaagaggaatggggtgccgcctgggacatgtacctgagtgccaatttggcgtcagagaagtccgtgctcctctcggcactcggatgcacggaggaagtctggttgctggcaagttacgacaccttcgcctccctcggcagcctcatcacctccgccacggccaagttcaacaccagagaggagaggaggcagctggaaagtttcatcgaggaaaaccaggacagcctctcctccgtcgctcgctccgtctcgcaagcgcttgaaaacacgaacaacaacattgcttggatggaagcaactatgacgtcatcgtccagtggctgaacgaccatggataggccagtga

SEQ ID NO: 4 - protein, 435 aa, Vibrio sp.
MLSATAGYGALRFLYLPSSFLFISSLLLPPLLLLHLTPLFIPLPRSLPSLSRLSTSLSFSYPSLPLSSSSLPLPYLALTQSSSSSTHHLHLPSALPHLTPPSQPPIPPLTLPPYILTSPLPPSISSSLSLLTHLPSHPPSISPRLSFPPLLLPSHPPSPSPHHPPSHPPLPPPHPPRSPPLILSQLHSTPHPSPHTLPPPSYPPTLTPPSSHSPLAHLPPHPRPPHFHLTPPPYFLPLPAIPLPSPAHSLYNSVGFEDDLQGPHLDQYKRAMALRWTCGLGYVDCVDRSVLQFEEWINNGSDVSPNLKSTVYCSAIAAGGEEEWGAAWDMYLSANLASEKSVLLSALGCTEEVWLLASYDTFASLGSLITSATAKFNTREERRQLESFIEENQDSLSSVARSVSQALENTNNNIAWMDSNYDVIVQWLNDHGYGQ

SEQ ID NO: 5 - DNA, 2072 bp, Vibrio sp.
atgcggtggacggtggccatcggcgggggcatcctggccatggccgtcctcatagggggggcggtctggggctacctggccaagcctcctcctgacgatctcttcaggctcccgacggatctgaagccggtccattacgaggtcagactccagcctttcctgagcgggaacttcagcgtgttgggtcacgtggacatcgagctgaaggcgctgacggaggcttacagtatcacgctgcatgtggctgacatcgatataaatactggcactatacgggtcgctccacccaactcaacgacagaaactgggttcgaaattttggaaacggcaaccgacacaaatctcgaccttttcgtggtgcacttgaaacaaagactcctcgagggtgagagttacatcttgtccttggacttcgagggtcacctcaacgacgagctgaggggcttctacaggtcgtcgtataaggatgaggctggggacgacaggatgctcgccgccaccttcttcgcgcccgcccacgcccgcagagccttcccgtgtatggacgagcccgccctcaaggccaccttctcgatctctctcgcccacgaggaccgcctgcacgccctctccaacatgccgctcagggactccgagccagtacatcccttcccccacgaacaccttcttcccaagtgcaatctagacgtgcaacaccctcctccctcctctaacccccgcccccccccttcgttgcagagaggggcaggcggggtgggtgtgggaccacttcgagacgtcggcgcccatgtccacgtacctcgtcgccttcgtcatctcggacttcaagagcaagtcgatggacgtgggcgagggaggccgccctcgagcaggtggactacgcccttgagaccggccctaaggcgttgtccttcttcgaggattacttcggcatcccttaccctcttcccaaggaggacatggtcgccttgccggacttcgctcccggggccatggagaactggggcctcatcacgtacagggagacggccatgctgtattcccccgaagagtcctccgcctcgaacaagcagcgggtggctacggttgtggtccacgagctcgcccaccagtggttcggaaacctcgtgacgccgacctggtggaccgacgtctggctcaacgagggttttgcgtccttcatggaatacgtcgggacggaaacgtcgaaccgtcctggcaaatgaaagaacagttcgttgtgtcggaacttcaatacgttttcgggatcgacagcctcgagtcgtctcatccaatcagcgttcccgtcgtgaaccaagatcagctgggcgagatctatgacgtcattgcttacgtcaaaggggcttcgattatccggatgatgaactattacctgggcgaagaaactttcagaaagggaatatcaaattacctcaaggctttcgaatacgccgcggccgaccaggacgacttgtggccagttcctgacacaggcggcgcacgaggacgacgccctggccgccgatgtcactgtgaaggacatcatggacacgtggacgctgcagacaggttacccggtcgtcaaggtggagagggacgtgacagggacgacggctcttgacgcccccgacttcacccggacgcgcccctcggcctggctgacccctggcacttccacgctcatcctcgacggccttccctccgccgacgcctgggtcctcctcaacctgcagcagacgggctacttccgggtcaactacgacgccggcaactgggagctcctgaccaagcagctggccgacgcccacgaggtcatccacgtcaccaaccgcgcgcaggtcatggacgacgccctcaacctcgcccgcgcagtgaaccccagctcccccacaacaaaccagctccctgaacctcagcttaccagccccttttttcacaaagccagctctcctgacctggctcacaaacccctgtttctccctcccccaaaaccccctcaccaacctttttccctccccctctcaccctctcaacaaaagctgctttctcctccaaaggccagcttcccctag

SEQ ID NO: 6 - protein, 690 aa, Vibrio sp.
MRWTVAIGGGILAMAVLIGGAVWGYLAKPPPDDLFRLPTDLKPVHYEVRLQPFLSGNFSVLGHVDIELKALTEAYSITLHVADIDINTGTIRVAPPNSTTETGFEILETATDTNLDLFVVHLKQRLLEGESYILSLDFEGHLNDELRGFYRSSYKDEAGDDRMLAATFFAPAHARRAFPCMDEPALKATFSISLAHEDRLHALSNMPLRDSEPVHPFPHEHLLPKCNLDVQHPPPSSNPRPPPSLQRGAGGVGVGPLRDVGAHVHVPRRLRHLGLQEQVGWTWAREAALEQVDYALETGPKALSFFEDYFGIPYPLPKEDMVALPDFAPGAMENWGLITYRETAMLYSPEESSASNKQRVATVVVHELAHQWFGNLVTPTWWTDVWLNEGFASFMEYVGTEHVEPSWQMKEQFVVSELQYVFGIDSLESSHPISVPVVNQDQLGEIYDVIAYVKGASIIRMMNYYLGEETFRKGISNYLKAFEYAAADQDDLWQFLTQAAHEDDALAADVTVKDIMDTWTLQTGYPVVKVERDVTGTTALDAPDFTRTRPSAWLTPGTSTLILDGLPSADAWVLLNLQQTGYFRVNYDAGNWELLTKQLADAHEVIHVTNRAQVMDDALNLARAVNPSSPTTNQLPEPQLTSPFFHKASSPDLAHKPLFLPPPKPPHQPFSLPLSPSQQKLLSPPKASFP

SEQ ID NO: 7 - DNA, 3325 bp, Vibrio sp.
atgcatcccgaagcggtcagccttgaaccttgtcgcgtcggccaaggcaggaaaggccgccgatctggggcgggaaggcctccagcgctccctctcccagcccccctcccctctgtcaaaacaaaccccctctcgaaccgggcgggcacgggccaccgggtactcgcgactggcccagtgtcgaactgtctcctgtcgggtgcggaggtgtgaccggaggatcggacgcgaagtgtataaccttgacgggcggagcgcaagtcgtgaccaggaacaaggaggactcgccgcctccgccgccatgaacaactacaacacgcaggcagcgacggacgtggtcgccatggacatccaccagcccgaccacatggtgtcctttgggaagaagaagggctgctacatcagccggagtgtgtcgctcctcctggccgtcttcttcctgagcgggatggtcgccacgggcctcctcgtctactactacgcgccgcacgacgtggaggccaaggcgcagcaggagaccatacgatatacgcaggcgaatgataacacgagaaatgttataccagaagttactaaaccacccaagatcaccacaaccactacaacgtcaacaaccacaaccactacaacaaagcccatgcctacaaccacccctactacaactacaaccacaatggctccaaaggagaaggtgaacgtacggttgcctcgctccctcaagccaatgcactacctggtcaaacttcagcctttaatcaacggcaacttcagcatcctcggctacgtggaggtggagatggaggtcctggagccgacctccaacatcacactccacatcgccgacattatcacttataatgatacagtgaaactaaagggaatggggaatgcatccgctccggggattaaaatgcatgaatacgacaactaccgagaattttacatcgctcacctggacaaggagctgcagcagggagagaagtacgtcctgtccatggaattcctcggttacctcaacgaccagctccgtggcttctacagatcctcatacaaggacgaagacgggaaagaaaaaatgcttgcggtgacacagttccaagccacgtctgcacgccgagccttcccttgcttcgacgagcccgcgctgaaggcaacgttcgaggtctacttgggacgccaggagaacatgtcatctatctccaacatgcgcattatggaaactatgcctattgagggtcaggagggctggctctgggaccactacgaggagagcgtccccatgtccacctacctcgtcgccttcgtcgtctcggatttcgccaacatgaactccactgtcaatgatcatgttctctttagggtgtggtcgcggcagtcggccatcaagcaggcagagtactcgagggagatcggtccggcgatcctcacgcacttcgaggactacttcggcgagccctacccgctccccaagcaggacatgatcgctatccccgacttctccgccggcgccatggagaactggggccttatcacatacagagagacggccatgctgtacgacccagtggtgtccgggccgagcaacaagcaccgggtggccctggtggtggcgcacgagttggcacaccagtggttcggtaacctcgtcactcccacgtggtggaccgacctgtggctcaatgaaggcttcgcatcgttcgttcagtacattggcatggattacgtcgaaccctcttggaaggtgatggaggagttcgttatctctcgactccagagggtgttcgccctcgacagcctcgagtcatctcacgagatcagcatccctgtcggtgcctctatcattcgtatgatgaaccacttccttagcgagaacactttcagaaagggagtaagcaactacctcactgcatttaagtacgaagccgccgagcaagatgacctgtgggagcacctcacgatggccgctcacgaggacggcactctgccccaggacgtcaccgtcaagaaggtcatggacacctggacgctgcagatgggataccccgtcatcaaggtggagaggagcgccgatggcatgtcagcctccgtctctcagaaccgtttcttgttggtggcgaaggaaaactcaagcgacgaccacgactacaagtggtgggttcctctcacctacacgactcagagcgaatccaacttcagtcagactcaagccatggtgtggatgaaggactccgaggaacagatcactctctcatctcttcccccgaaggacgagtgggtcatcttcaacctgcaggagacgggctactacagggtcaactacgacgaccacaactggggcctcctcatccagcagctgaaggacgaccacgaggtcatcagcaccaccaaccgagcccagattatcgacgacgcgatggacctcgctcgtgctggccagctgaactatgagatcgcccttggggtctacgcttacttaggcaacgagacagaatacgtgccctgggcagctgctgttaacaatattggttatctggaaggcatgttcaaacgcaaggccggatatggggctctcaagaaatacatcctggacctggtggtgccgctgtacgagtccgtcggcttcaccaacaggcacgacgacccattcctggagcagtcgaagcgcaggacggccgtgtcttgggcgtgtatgctcggccatcaggactgcctcgataacgtcctctcactgtaccgacagtggatgagcaatcccgagaacgagaccctcatctcgcctaacctgaaatcgacggtctactgccgcgccatcgccgagggcggggaggccgagtgggacttcgcgtgggatcagtacctcaagtccaacgtgggcaccgagaaggccctcctgctctctgccatgggctgcagcaaggagatctggatcttgtcaaggtacctggacatggctttcacgcccggcagcggcatcaggaagcaggactccgaccgcgtcttcgcctcggtggcttacaacaaggtcggcggccccctggcgtggcgcttcctcagggaccagtggaagagaatctatgacttccatggaaaacccaagggcggcctcatcaaatccggaacctcaggcttcaacacagacctgcagctgaaggagatcgaactgttcaaacaagaacacgaagaggaactgggtggcgtctcccgaagcgtcgaccaagttctcgaaagcacaaagaacagcatcgcttggcttgaccgcaattacgagaccatcgtccagtggcttgataacaacggctacagcaccaaactccagaacgagtag

SEQ ID NO: 8 - protein, 1106 aa, Vibrio sp.
MHPEAVSLEPCRVGQGRKGRRSGAGRPPALPLPAPSPLSKQTPSRTGRARATGYSRLAQCRTVSCRVRRCDRRIGREVYNLDGRSASRDQEQGGLAASAAMNNYNTQAATDVVMDIHQPDHMVSFGKKKGCYISRSVSLLLAVFFLSGMVATGLLVYYYAPHDVEAKAQQETIRYTQANDNTRNVIPEVTKPPKITTTTTTSTTTTTTTKPMPTTTPTTTTTTMAPKEKVNVRLPRSLKPMHYLVKLQPLINGNFSILGYVEVEMEVLEPTSNITLHIADQITYNDTVKLKGMGNASAPGIKMHEYDNYREFYIAHLDKELQQGEKYVLSMEFLGYLNDQLRGFYRSSYKDEDGKEKMLAVTQFQATSARRAFPCFDEPALKATFEVYLGRQENMSSISNMRIMETMPIEGQEGWLWDHYEESVPMSTYLVAFVVSDFANMNSTVNDHVLFRVWSRQSAIKQAEYSREIGPAILTHFEDYFGEPYPLPKQDMIAIPDFSAGAMENWGLITYRETAMLYDPVVSGPSNKHRVALVVAHELAHQWFGNLVTPTWWTDLWLNEGFASFVQYIGMDYVEPSWKVMEEFVISRLQRVFALDSLESSHEISIPVGASIIRMMNHFLSENTFRKGVSNYLTAFKYEAAEQDDLWEHLTMAAHEDGTLPQDVTVKKVMDTWTLQMGYPVIKVERSADGMSASVSQNRFLLVAKENSSDDHDYKWWVPLTYTTQSESNFSQTQAMVWMKDSEEQITLSSLPPKDEWVIFNLQETGYYRVNYDDHNWGLLIQQLKDDHEVISTTNRAQIIDDAMDLARAGQLNYEIALGVYAYLGNETEYVPWAAAVNNIGYLEGMFKRKAGYGALKKYILDLVVPLYESVGFTNRHDDPFLEQSKRRTAVSWACMLGHQDCLDNVLSLYRQWMSNPENETLISPNLKSTVYCRAIAEGGEAEWDFAWDQYLKSNVGTEKALLLSAMGCSKEIWILSRYLDMAFTPGSGIRKQDSDRVFASVAYNKVGGPLAWRFLRDQWKRIYDFHGKPKGGLIKSGTSGFNTDLQLKEIELFKQEHEEELGGVSRSVDQVLESTKNSIAWLDRNYETIVQWLDNNGYSTKLQNE

SEQ ID NO: 9 - DNA, 2966 bp, Vibrio sp.
atgagcggctcgagcagagaagtcctcgccatggaaacgagccacccggaccacgtgatctccttcgggaagaagaagggctgctacgtgagtcgctgcgtggcggccctgctcggcgtcttcttcctgagcgggatggtcgccacgggcctcctcgtctactactacgcgccgcacatccgggactcgcagcgggagtcgttgattctgcagaagcctgtggtcccgctgcacaagtccctgccgccgccgacgagaaggccctccgccacgtcaacgaccacggaggccctcagacccacagtccagcccccgacgacctccgccacagcagcccccgccgcagaggccctggacgtgcgactgccgacggcgctgaggccgcttcactacctgatcaaacttcagcccttcatcaacgggaacttcagcatcctgggctacatggaggtggaaatggaagtcttggaaccaacgtccaacatcacgctgcacatcgctgacatcatcacccataatgatactgttactgtggcggcctcgggagactcgggtcccagcataaggataaagaggcaccagtacgaccaacgaccgccagttctacatcgcgcagctggatcagcaactagagaagaacaagaagtacgtgctgtccatggagttcctgggttacctcaacgaccagctccgcggattctacaggtccacttataaggacgaagatggaagcgataagatgctggccgtgacccagttccaggccacggacgcgcgccgggccttcccctgcttcgacgagcccgagatgaaggcgacgttcgaagtgtccttggccagggaggagaatatgtcgtccatctcgaatatgccgatcaaggagactctgccagttcaaaaccaggagggatgggtatgggaccattaccatagaagtgttccaatgtctacttacctggttgctttcgtggtgtccgactttgccaacctaaagtctcgagctaatgagaacaccttcttccgagtgtgggcgagagagtcggcgatccagcaggccgagtacgcgggccaggtgggtcccatgatcctgaaccacttcgaaaagtacttcagcatgccctacccgctgcccaaacaggacatgatcgccatcccggacttctcggcgggagcgatggagaactggggcctcattacctacagagaaacggcgatgctttacgaccccgtggtgtcagcggcttccaacaagcagtatgtggtggccgttgtggcgcacgagttggcgcatcagtggtttggcaacatcgtcacgccgagctggtggactgacctctggctcaatgagggctttgcttcctacgtcgagtacataggcatcaaccatgtcgagcccaagtggcaggtgatggagcagttcgtgctgcgggaggtgcaggaggtcttcgggctggactgcctcgagtcctcgcacccgatcagcatccccgtcgggcatccggacgaaattggacaaatattcgacaggatttcttacggcaaaggtgcatctataatccgcatgatgaaccacattcctcactgaggtcaccttcagaagaggcctaaggaactatctcgatgcttttaagtacagcacggcggaacaggacgacttgtgggaatacctgaccgccgtcgcgcaccaggacggaacgctgcctcgaggtctgacggtgaagatgatcatggacacgtggacgctgcagatgggctaccccgtcgtgaaggtgacgcggggccccgacggcacctcggcagttgtatcgcaggaacgttttctcttggtgagaagcgaaaactccagcgacacccacgactacaagtggtgggttcccctgacgtacacgacccagagcgaggccaacttcaaccagactcaggccatggtgtggatgaaggactccgaggcacagatcagcctctcgtcccttcccccgagggaccagtgggtcatcttcaacctgcaggagacgggctactacagggtcaactacgacgaccacaactggggcctcctcatccagcagctgaggaacgaccacgaggtcatcagcaccatcaacagagcgcagatcatcgacgacgccatgaatcttgccaaagctggtcagattacgtacgaaaccgctctaagcgtgtatacttacctgtcgaaggagacggagtatgtgcccttggctgctgccatcaacaaccttggctacctgaggagcatgttcgtgcgggcaggtggctatggctcgctcaggagctacctgctggacatcctggtgccgctctacgagtccgtgggcttcgaggacagccccgacgaccctctgctggaccagtacaagcggacgaaggcgctctcgtgggcttgcctcttgggccaccagcactgcctggacagcgcctccgccctctaccgcacctggatggccaacccgaccaacgacagcatcatttccccgaacctcaagtcgacggtttactgccgcgccatcgcagagggtggtgaagccgagtggaactttgcttggcataaatatctcaagtacttagagatggccttcactcccgactctggcatccggaaacaagacgcttaccgcgtgttcggagccgtcgccaagaacgtggtgggtcgcccgctcgcctggaactaccttcagaatgagtgggacaaaatatacgacttttacgggaaggcgaaaccccacttcataaagtacgccactggaggcttcaacacggagcagcacttgaaagaggtggagcacttccgaaaggagcacgagcaccacctgggctccgcgtcgcgcacggtcgagcaggtgatcgagcggacgaagaacaacatcgcctggatgaagaccaactatgacgtcatcgtgaagtggctggacgcgaacgggtattcgacgaagctcagcacggcatag

SEQ ID NO: 10 - protein, 987 aa, Vibrio sp.
MSGSSREVLAMETSHPDHVISFGKKKGCYVSRCVAALLGVFFLSGMVATGLLVYYYAPHIRDSQRESLILQKPVVPLHKSLPPPTRRPSATSTTTEALRPTVQPPTTSATAAPAAEALDVRLPTALRPLHYLIKLQPFINGNFSILGYMEVEMEVLEPTSNITLHIADIITHNDTVTVAASGDSGPSIRIKRHQYDHDRQFYIAQLDQQLEKNKKYVLSMEFLGYLNDQLRGFYRSTYKDEDGSDKMLAVTQFQATDARRAFPCFDEPEMKATFEVSLAREENMSSISNMPIKETLPVQNQEGWVWDHYHRSVPMSTYLVAFVVSDFANLKSRANENTFFRVWARESAIQQAEYAGQVGPMILNHFEKYFSMPYPLPKQDMIAIPDFSAGAMENWGLITYRETAMLYDPVVSAASNKQYVVAVVAHELAHQWFGNIVTPSWWTDLWLNEGFASYVEYIGINHVEPKWQVMEQFVLREVQEVFGLDCLESSHPISIPVGHPDEIGQIFDRISYGKGASIIRMMNHFLTEVTFRRGLRNYLDAFKYSTAEQDDLWEYLTAVAHQDGTLPRGLTVKMIMDTWTLQMGYPVVKVTRGPDGTSAVVSQERFLLVRSENSSDTHDYKWWVPLTYTTQSEANFNQTQAMVWMKDSEAQISLSSLPPRDQWVIFNLQETGYYRVNYDDHNWGLLIQQLRNDHEVISTINRAQIIDDAMNLAKAGQITYETALSVYTYLSKETEYVPLAAAINNLGYLRSMFVRAGGYGSLRSYLLDILVPLYESVGFEDSPDDPLLDQYKRTKALSWACLLGHQHCLDSASALYRTWMANPTNDSIISPNLKSTVYCRAIAEGGEAEWNFAWHKYLKYLEMAFTPDSGIRKQDAYRVFGAVAKNVVGRPLAWNYLQNEWDKIYDFYGKAKPHFIKYATGGFNTEQHLKEVEHFRKEHEHHLGSASRTVEQVIERTKNNIAWMKTNYDVIVKWLDANGYSTKLSTA

SEQ ID NO: 11 - protein, 5 aa, Artificial Sequence
  Synthetic GAMEN motif
GAMEN

SEQ ID NO: 12 - protein, 24 aa, Artificial Sequence
  Synthetic zinc-binding motif
  misc_feature (3)..(4): Xaa can be any naturally occurring amino acid
  misc_feature (6)..(23): Xaa can be any naturally occurring amino acid
HEXXHXXXXXXXXXXXXXXXXXXE

SEQ ID NO: 13 - protein, 15 aa, Artificial Sequence
  Synthetic linker
GGGGGGGGGGGGGGG

SEQ ID NO: 14 - protein, 15 aa, Artificial Sequence
  Synthetic linker
GGGGSGGGGSGGGGS

SEQ ID NO: 15 - protein, 5 aa, Artificial Sequence
  Synthetic ligand
TEFCA

SEQ ID NO: 16 - protein, 149 aa, Artificial Sequence
  Synthetic consensus sequence
  misc_feature (2)..(3): Xaa can be any naturally occurring amino acid
  misc_feature (10)..(10): Xaa can be any naturally occurring amino acid
  misc_feature (21)..(21): Xaa can be any naturally occurring amino acid
  misc_feature (36)..(36): Xaa can be any naturally occurring amino acid
  misc_feature (46)..(46): Xaa can be any naturally occurring amino acid
  misc_feature (53)..(53): Xaa can be any naturally occurring amino acid
  misc_feature (56)..(57): Xaa can be any naturally occurring amino acid
  misc_feature (59)..(59): Xaa can be any naturally occurring amino acid
  misc_feature (61)..(61): Xaa can be any naturally occurring amino acid
  misc_feature (63)..(63): Xaa can be any naturally occurring amino acid
  misc_feature (65)..(65): Xaa can be any naturally occurring amino acid
  misc_feature (70)..(70): Xaa can be any naturally occurring amino acid
  misc_feature (72)..(72): Xaa can be any naturally occurring amino acid
  misc_feature (75)..(75): Xaa can be any naturally occurring amino acid
  misc_feature (79)..(79): Xaa can be any naturally occurring amino acid
  misc_feature (85)..(86): Xaa can be any naturally occurring amino acid
  misc_feature (89)..(89): Xaa can be any naturally occurring amino acid
  misc_feature (98)..(98): Xaa can be any naturally occurring amino acid
  misc_feature (110)..(110): Xaa can be any naturally occurring amino acid
  misc_feature (118)..(118): Xaa can be any naturally occurring amino acid
  misc_feature (123)..(123): Xaa can be any naturally occurring amino acid
  misc_feature (125)..(125): Xaa can be any naturally occurring amino acid
SXXRLPTTLXPLHYLVKLQPXINGNFSFLGYVEIEXEVLEATSNIXLHIADIXIQXXTXKXAXGXNVGIXIXTHXYDNXRQFLRXXLDXELLVGNKYXLSMDFEGYLNDXLRGFYRSXYKDEXGXDRMLATTQFQPTHARRAFPCFDEP

SEQ ID NO: 17 - protein, 161 aa, Bombyx mori
LRDPAFRLPTTTRPRHYQVTLTPYFDVVPANVNPFTFDGEVTIYTSPTVANVNEVVIHCNDLTIQSLSIGYQSGTNVVDITATGQTFACEMPFSFLRIRTTEALVLNREYIIKSTFRGNLQTNMRGFYRSWYVDSTGRRWMGTTQFQPGHARQAFPCYDEP

SEQ ID NO: 18 - protein, 148 aa, Bombyx mori
SQYVLPGESFPTFYDVSLFIDPANTVSFNGKVSIRIIPRIATNVIVVQAMEMTIRSISVFTDRNSNENLFTSFTLATDDTHFLRISTRTQLLPDQPYIVNIDYESKYAPNMFGVYVSTYQQNGRTVNLVTSQLQPTFARRAFPCYDEP

SEQ ID NO: 19 - protein, 154 aa, Bombyx mori
IAASTYRLPTTTRPLHYNVLWAIDISRLTFSGTVEIQLYATRANVSEIVIHADDLEITSVILRQGTVTTPSTYTLQKELQFLRLRLNTGTLVFNAASPVIYTLTIDFAARLRTDMYGIYRTWFRNSANDVTRWMASTQFQATSARYAFPCYDEP

SEQ ID NO: 20 - protein, 153 aa, Bombyx mori
VAESAYRLLDTIQPRTMRVDLDVFLNEARFDGIVSMDIEVLASNIEQIVFHQNVVSIQGVNLVTARGDPVGLKFPDPFTIDRHYELLLINLAQPIAAGNYTVTVRYRGQINTNPVDRGFYRGYYYVNNQLRYYATTQFQPFHARKAFPCFDEP

SEQ ID NO: 21 - protein, 151 aa, Penaeus vannamei
IDVRLPRSVKPLHYKVKLQPLINGNFSILGYVEVEVEVLEATSNVTLHIADIVTKNETIKLAPSDQVQGPGIGINKHSYDNERQFYVAELGEELEVGKKYVLSMDFEGYLNDQLHGFYSRLTRRGRSDRLIASTQFQPTDARRAFPCFDEP

SEQ ID NO: 22 - protein, 150 aa, Penaeus vannamei
FRLPTDLKPVHYEVRLQPFLSGNFSVLGHVDIELKALTEAYSITLHVADIDINTGTIRVAPPNSTTETGFEILETATDTNLDLFVVHLKQRLLEGESYILSLDFEGHLNDELRGFYRSSYKDEAGDDRMLAATFFAPAHARRAFPCMDEP

SEQ ID NO: 23 - protein, 152 aa, Penaeus vannamei
EKVNVRLPRSLKPMHYLVKLQPLINGNFSILGYVEVEMEVLEPTSNITLHIADIITYNDTVKLKGMGNASAPGIKMHEYDNYREFYIAHLDKELQQGEKYVLSMEFLGYLNDQLRGFYRSSYKDEDGKEKMLAVTQFQATSARRAFPCFDEP

SEQ ID NO: 24 - protein, 154 aa, Penaeus vannamei
AEALDVRLPTALRPLHYLIKLQPFINGNFSILGYMEVEMEVLEPTSNITLHIADIITHNDTVTVAASGDSGPSIRIKRHQYDHDRQFYIAQLDQQLEKNKKYVLSMEFLGYLNDQLRGFYRSTYKDEDGSDKMLAVTQFQATDARRAFPCFDEP

SEQ ID NO: 25 - protein, 60 aa, Artificial Sequence
  Synthetic consensus sequence
  misc_feature (51)..(51): Xaa can be any naturally occurring amino acid
GAMENWGLITYRETAMLYDPVVSNASNKQRVATIVAHELAHQWFGNLVTPXWWTDLWLNE

SEQ ID NO: 26 - protein, 60 aa, Bombyx mori
GAMENWGLLTYREALILYDPLNSNHFYKQRVANIVAHEIAHMWFGNLVTCAWWDNLWLNE

SEQ ID NO: 27 - protein, 60 aa, Bombyx mori
GAMENWGLVIYREVALLVTEGVTTTATRQNIARIICHENVHMWYGNEVGPLSWTYTWLNE

SEQ ID NO: 28 - protein, 60 aa, Bombyx mori
GAMENWGLLTYREAYLMYDENHTNGYFKQLIAYILSHEIAHMWYGNLVTCDWWDVLWLNE

SEQ ID NO: 29 - protein, 60 aa, Bombyx mori
GAMENWGMVNYREAYLLYDPQHTNLINKIFIATIMAHELAHKWFGNLVTCFWWSNLWLNE

SEQ ID NO: 30 - protein, 60 aa, Penaeus vannamei
GAMENWGLITYRETAMLYDPAVSAASNKQRVVVVVAHELAHQWFGNLVTPEWWTDLWLNE

SEQ ID NO: 31 - protein, 52 aa, Penaeus vannamei
SSSSSTHHLHLPSALPHLTPPSQPPIPPLTLPPYILTSPLPPSISSSLSLLT

SEQ ID NO: 32 - protein, 60 aa, Penaeus vannamei
GAMENWGLITYRETAMLYSPEESSASNKQRVATVVVHELAHQWFGNLVTPTWWTDVWLNE

SEQ ID NO: 33 - protein, 60 aa, Penaeus vannamei
GAMENWGLITYRETAMLYDPVVSGPSNKHRVALVVAHELAHQWFGNLVTPTWWTDLWLNE

SEQ ID NO: 34 - protein, 60 aa, Penaeus vannamei
GAMENWGLITYRETAMLYDPVVSAASNKQYVVAVVAHELAHQWFGNIVTPSWWTDLWLNE

(19) United States
(12) Patent Application Publication
(10) Pub. No.: US 2024/0002457 A1
(43) Pub. Date: Jan. 4, 2024

(54) VIBRIO TOXIN BINDING PROTEINS IN SHRIMP AND USES THEREOF

(71) Applicant: ARIZONA BOARD OF REGENTS ON BEHALF OF THE UNIVERSITY OF ARIZONA, Tucson, AZ (US)

(72) Inventors: Hung Mai, Tucson, AZ (US); Arun Dhar, Tucson, AZ (US)

(21) Appl. No.: 18/037,098

(22) PCT Filed: Nov. 16, 2021
(86) PCT No.: PCT/US2021/059561

§ 371 Date: May 16, 2023

Related U.S. Application Data
(60) Provisional application No. 63/114,383, filed on Nov. 16, 2020.

(51) Int. Cl. C07K 14/435; A23K 20/147; A23K 20/189; A23K 50/80; C12N 9/16; C12N 9/48; C12Q 1/6883

(52) U.S. Cl. CPC C07K 14/43509; A23K 20/147; A23K 20/189; A61K 38/00; C12N 9/16; C12N 9/485; C12Q 1/6883; A23K 50/80

(57) ABSTRACT

Described herein are proteins and fragments thereof that can be capable of binding or otherwise interacting with a Vibrio species toxin. The Vibrio toxin binding proteins and fragments thereof can be included in a formulation, such as a feed formulation, that can be used to treat and/or prevent Vibrio spp. disease or a symptom thereof, such as AHPND. Also described herein are AHPND susceptibility signatures that can be useful for identification of susceptible and/or resistant/tolerant organisms.

### CROSS-REFERENCE TO RELATED APPLICATIONS

This application claims the benefit of and priority to co-pending U.S. Provisional Patent Application No. 63/114,383, filed on Nov. 16, 2020, entitled “VIBRIO TOXIN BINDING PROTEINS IN SHRIMP AND USES THEREOF,” the contents of which is incorporated by reference herein in its entirety.

### SEQUENCE LISTING

This application contains a sequence listing filed in electronic form as an ASCII.txt file entitled UAZ-0130WP_ST25.txt, created on Nov. 16, 2021, and having a size of 74,963 bytes. The content of the sequence listing is incorporated herein in its entirety.

### TECHNICAL FIELD

The subject matter disclosed herein is generally directed to treatment and prevention of hepatopancreatic necrosis disease in shellfish, such as shrimp, as well as detecting and developing susceptible and resistant strains of animals.

### BACKGROUND

Fish and shellfish serve as a major source of revenue in many countries with large coastal boundaries in Asia and the Americas, which are largely responsible for the global aquaculture production. Additionally, seafood serves as a major source of protein in these countries. Among various aquaculture species, shrimp is a high value species that ranks second position in contributory value with a total of about $142 billion. Despite the importance of shrimp to the global economy and food source, shrimp farming has often been accomplished on the background of poor biosecurity measures and lack of attention to biosecurity warnings. As an inevitable result, the list of deadly diseases in shrimp aquaculture has been growing since the first report of a viral disease in shrimp, Baculovirus penaei (BP). As such there exists a need for the development of compositions, methods, and techniques for detection, prevention, treatment, and/or control of diseases, such as acute hepatopancreatic necrosis disease (AHPND), affecting aquaculture, and more particularly, crustacean aquaculture.

Citation or identification of any document in this application is not an admission that such a document is available as prior art to the present invention.

### SUMMARY

Described in certain example embodiments herein are a Vibrio spp. toxin binding protein, wherein the Vibrio spp. toxin binding protein is capable of binding or otherwise interacting with a Vibrio spp. toxin.

In certain example embodiments, the Vibrio spp. toxin is PirA, PirB, a PirA-like toxin, a PirB-like toxin, or a combination thereof.

In certain example embodiments, the Vibrio spp. toxin binding protein is aminopeptidase-N or a fragment thereof, alkaline phosphatase (ALP) or a fragment thereof, midgut membrane-bound cadherin (CAD) or a fragment thereof, or a combination thereof.

In certain example embodiments, the Vibrio spp. toxin binding protein comprises a cry protein binding region (CBR), a GAMEN (SEQ ID NO: 11) motif, a Zn²⁺ binding region, or a combination thereof.

In certain example embodiments, the Vibrio spp. toxin binding protein is a recombinant protein.

In certain example embodiments, the Vibrio spp. toxin binding protein comprises a polypeptide that is 50-100% identical to any one of SEQ ID NO: 2, 4, 6, 8, or 10 or is a fragment thereof of at least 3 amino acids.

Described in certain example embodiments herein are formulations, comprising a Vibrio spp. toxin binding protein or a fragment thereof of any one of the preceding paragraphs; and a suitable carrier.

In certain example embodiments, the formulation is a feed formulation suitable for a crustacean.

In certain example embodiments, the crustacean is a shrimp.

In certain example embodiments, the formulation is adapted for delivery in a water source.

In certain example embodiments, the formulation is effective to treat or prevent an infection, disease, or a symptom thereof of a Vibrio spp. organism in a shrimp.

In certain example embodiments, the disease is AHPND.

Described in certain embodiments herein are polynucleotides that encode any one of the Vibrio spp. toxin binding protein or a fragment thereof of any one of the preceding paragraphs.

Described in certain embodiments herein are vectors or vector systems that include a polynucleotide of the preceding paragraph.

Described in certain embodiments herein are cells comprising a polynucleotide and/or a vector or vector system of any of the preceding paragraphs.

In certain example embodiments, the cell is capable of producing the Vibrio spp. toxin binding protein.

Described in certain example embodiments, are methods of treating or preventing a Vibrio spp. infection or disease comprising administering an amount of a protein, vector or vector system, cell, or formulation as in any one of the preceding paragraphs and elsewhere herein to a subject.

In certain example embodiments, the subject is a non-human animal.

In certain example embodiments, the is a crustacean.

In certain example embodiments, the subject is a shrimp.

In certain example embodiments, administrating is via a feed or water source.

In certain example embodiments, the subject is infected with, is suspected of being infected with an organism of a Vibrio spp., or will be exposed to an organism of a Vibrio spp.

Described in certain example embodiments herein are methods of detecting an acute hepatopancreatic necrosis disease (AHPND) and/or AHPND susceptible organisms and/or cells therefrom comprising detecting in one or more cells an AHPND susceptibility signature, wherein the AHPND tolerance signature comprises: one or more genes selected from APN, ALP, or CAD; and/or one or more SNPs in one or more genes selected from APN, ALP, or CAD; wherein detecting of the susceptibility signature indicates that the organism and/or cell is tolerant/resistant or susceptible to AHPND.

In certain example embodiments, the organism is a crustacean.

In certain example embodiments, the organism is a crab, lobster, crayfish, shrimp, prawn, or krill.

In certain example embodiments, the organism is obtained from a cell, an organ, a tissue, a bodily fluid, or a combination thereof.

In certain example embodiments, the sample is obtained from a hepatopancreas of the organism.

Described in exemplary embodiments herein are methods of treating or preventing a Vibrio spp. infection or disease comprising detecting an AHPND susceptible organism by performing a method as described as in any one of the preceding paragraphs and elsewhere herein; and performing a method as in any one of the preceding paragraphs.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—The distribution of AHPND in the world (modified from Zorriehzahra and Banaederakhshan, 2015). The red color indicated the presence of AHPND. AHPND was first reported in China in 2009. Since then it was reported in several shrimp producing countries including Vietnam (2010), Malaysia (2011), Thailand (2012), Mexico (2013), US (2017), Bangladesh (2017).

FIGS. 2A-2B—Clinical signs and etiological agent of acute hepatopancreatic necrosis disease (AHPND). (FIG. 2A) Clinical sign of AHPND in Penaeus vannamei. The animal on the left represents a healthy animal and the animal on the right represents a AHPND affected animal. The hepatopancreas in the healthy animal is dark brown and the gut is full whereas in diseased animal, the hepatopancreas is pale and the gut is empty (Taken from Kaneshamoorthy et al., 2020). (FIG. 2B) A schematic representation of Vibrio parahaemolyticus cell (left) displaying genomic content, two chromosomes and the plasmid DNAs. A genetic map of the 70.453 kb plasmid DNA containing the binary toxin genes, pirA and pirB is shown on the right (Taken from Lee et al., 2014).

FIGS. 3A-3C—Histopathology of hepatopancreases (HP) collected from AHPND-infected shrimp and healthy shrimp. (FIG. 3A) The HP collected from healthy shrimp showed normal structure of tubule and epithelial cells in the hepatopancrease including R-cells (arrow) and B-cells (FIG. 3B) (arrow). Hepatopancreas tissue section from AHPND infected animal displaying acute phase infection characterized by the atrophy of hepatopancreatic tubule, depletion of B- and R-cells, and sloughing of HP tubule epithelial cells and remnants of necrotic cells in the lumen of the HP tubule (arrow) (FIG. 3C) the AHPND-infected HP displaying the chronic phase infection which resemble to septic hepatopancreatic necrosis (SHPN) (arrow) showed only a few tubules with epithelial necrosis accompanied by bacteria and inflammation.

FIGS. 4A-4B—A comparison of PirAB^{VP} structure with structure of Cry toxin released by Bacillus thuringiensis. (FIG. 4A) The protein structure of PirA^{VP} and PirB^{VP} (Taken from Lin et al., 2017). The PirA^{VP} has beta sheet in structure. PirB^{VP} contains alpha-helix in N-terminal and Beta sheet in C-terminal in structure. (FIG. 4B) The similarity in structure between PirAB^{VP} and Cry toxin. The PirA shares similar structure with Cry domain III. The PirB^{VP} N-terminal shares similar structure with Cry domain I and PirB^{VP} C-terminal structure shares similarity to Cry domain II. The shared structure of PirAB^{VP} and Cry toxin suggested that the mode action of PirAB in shrimp might be similar to mode action of Cry toxin in insects.

FIGS. 5A-5B—A predicted structure of PirA^{VP}/PirB^{VP} toxin of Vibrio parahaemolyticus causing AHPND. (FIG. 5A) The structure of PirA^{VP} were built using Swiss-model (https://swissmodel.expasy.org/interactive). (FIG. 5B) The structure of PirB^{VP} were built using Swiss-model (https://swissmodel.expasy.org/interactive). The Global Model Quality Estimation (GMQE) values of proposed PirA models ranged from 0.92-0.93. The Qualitative model energy analysis (QMEAN) value of the proposed PirA^{VP}/PirB^{VP} models ranged from −0.37 to −0.91. The QMEAN and GMQE values are used as a criteria to determine the quality of a predicted tertiary structure. The GMQE value ranges from 0 to 1.0. A GMQE value close to 1.0 indicates the high quality of a model. A QMEAN value close to 0 indicates the proposed model is reliable. The “thumb up” sign in QMEAN indicates an acceptable value.

FIG. 6—A predicted PirAB^{VP} structure was generated using the Gramm-X server (http://vakser.compbio.ku.edu/resources/gramm/grammx/) (Tovchigrechko et al 2006). The binary toxin contains four subunits, two of PirA and two of PirB. The interaction interface (dotted line) between two heterodimer (PirA^{VP}/PirB^{VP}) occurred between two PirA molecules. Arrows indicated the PirB^{VP}, arrows indicated PirAB^{VP}. First, the monomer PirA^{VP} and PirB^{VP} models were built using SWISS-model. Then a heterodimers of PirA^{VP} and PirB^{vp} models were built using Gramm-X server (http://vakser.compbio.ku.edu/resources/gramm/grammx/) (Tovchigrechko et al 2006). Then, a tetraheteromer between two heterodimers of PirA^{VP} and PirB^{vp} was generated using the Gramm-X server. (http://vakser.compbio.ku.edu/resources/gramm/grammx/) (Tovchigrechko et al 2006).

FIGS. 7A-7E—Aminopeptidase N (APN) transmembrane protein of Penaeus vannamei shrimp. The amino acid (aa) sequence of five isoforms of APN were obtained from the GenBank database (GenBank Accession No. of APN-1 is ROT67356.1; APN-2, ROT67357.1; APN-3, ROT67358.1, APN-4, ROT77087.1; APN-5, ROT72064.1). The predicted transmembrane domain was determined using TMHMM server v2.0 (http://www.cbs.dtu.dk/services/TMHMM/). The results revealed the presence of a transmembrane domain in the 5′-end of the polypeptide for each of the five APNs. APN-1 included intracellular region (aa 1 to 33), transmembrane domain (34^{th}aa-56^{th} aa), extracellular region (57^{th}aa-978^{th} aa). APN-2 includes an intracellular region (1^{st}aa-12^{th} aa), transmembrane domain (13^{th}aa-35^{th} aa), extracellular region (36^{th}aa-435^{th} aa). APN-3 included intracellular region (1^{st}aa-4^{th} aa), transmembrane domain (5^{th}aa-27^{th} aa), extracellular region (28^{th}aa-690^{th} aa). APN-4 included intracellular region (1^{st}aa-137^{th} aa), transmembrane domain (138^{th}aa-160^{th} aa), extracellular region (161^{th}aa-1107^{th} aa). APN-5 included intracellular region (1^{st}aa-33^{rd} aa), transmembrane domain (34^{th}aa-56^{th} aa), extracellular region (57^{th}aa-987^{th} aa). In each panel of APN, the blue color indicates the intracellular region, red color indicates a transmembrane domain, and pink color indicates extracellular domain.

FIG. 8—Multiple alignment of Aminopeptidase N Transmembrane protein of Penaeus vannamei shrimp with APN in silkworm Bombyx mori. The amino acid sequences of APN of silkworm and shrimp were obtained from the GenBank and aligned using a multiple alignment tool, CLUSTAL W. TM=Transmembrane, CBR=Cry Binding Region. The result shows that APNs in shrimp contain a (BR similar to APN in silkworm. The conserved residues in CBR are highlighted in black. The amino acid residues GAMEN (SEQ ID NO: 11) and HEXXHX¹⁸E (SEQ ID NO: 12) zinc-binding motifs are highly conserved in APN isoforms in shrimp. The presence of CBR and highly conserved motifs in active site of APNs in shrimp indicate that APNs in shrimp serve as receptors of PirAB^{VP} toxin.

FIG. 9—Phylogenetic tree of Aminopeptidase N Transmembrane protein of Penaeus vannamei shrimp with APN in other species. Px=Plutella xylostella; Tn=Trichoplusia ni; Bm=Bombyx mori; Mb=Mamestra brassicae; Aa=Aedes aegypti; Ld=Lymantria dispar; Ag=Aphis glycines; Ms=Manduca sexta; Ap=Acyrthosiphon pisum; Nl=Nilaparvata lugens; Pv=Penaeus vannamei. The APNs from Penaeus vannamei was a separated group suggesting APNs in shrimp may play may have different function in PirAB^{VP} binding. The pylogenetic tree was generated by using MEGA-X program with Maximum likely hood, boostrap replicates of 1000.

FIG. 10—A predicted tertiary structure of amino peptidase-N isoform 1 (APN-1) in shrimp (Penaeus vannamei). The predicted structure was generated by using a SWISS-Model (https://swissmodel.expasy.org/interactive). The APN-1 contains alpha helix and beta sheet in its tertiary structure. The GMQE and QMEAN values were 0.75 and −3.27, respectively. The model contains one Zinc ion in structure. The acceptable values of GMQE and QMEAN indicate the proposed model has a structure close to the native structure of APN-1 in vivo.

FIG. 11—A predicted model of interaction between PirA^{VP} and APN-1. The model was generated by using the Gramm-X server (http://vakser.compbio.ku.edu/resources/gramm/grammx/) (Tovchigrechko et al 2006). The structures of PirA^{VP} and APN-1 were built by using SWISS-model (https://swissmodel.expasy.org/interactive). The built structures of PirA^{VP} and APN-1 was submitted to Gramm-X server for modelling of interaction. Ten proposed models of interaction between PirA^{VP} and APN-1 were generated. Only one out of ten models showed the interaction between PirA^{VP} and CBR on APN-1, suggesting this model is the mode of interaction between PirA^{VP} and APN-1 fin shrimp. The red-dashed circle indicated the proposed interaction region.

FIG. 12—A proposed model of interaction between PirB^{VP} and APN-1. The model was generated by using the Gramm-X server (http://vakser.compbio.ku.edu/resources/gramm/grammx/) (Tovchigrechko et al 2006). The structures of PirB^{VP} and APN-1 were built by using SWISS-model (https://swissmodel.expasy.org/interactive). The predicted structures of PirB^{VP} and APN-1 were submitted to the Gramm-X server for modelling of interaction(s). Ten proposed models of interaction between PirB^{VP} and APN-1 were generated. All ten models revealed that PirB^{VP} contacted to the helix region of the predicted tertiary structure of APN-1. None of the ten models showed any interaction between PirB^{VP} and CBR in APN-1, suggesting PirB^{VP} is not involved in the interaction of PirAB^{VP} and APN-1 in vivo.

FIG. 13—A model of interaction between PirAB^{VP} and APN-1. The model was generated by using the Gramm-X server (http://vakser.compbio.ku.edu/resources/gramm/grammx/) (Tovchigrechko et al 2006). The structures of PirAB^{VP} and APN-1 were built by using the Gramm-X and SWISS-model (https://swissmodel.expasy.org/interactive). The predicted tertiary structures of PirAB^{VP} and APN-1 were submitted to Gramm-X server for modelling of interaction(s). Ten proposed models of interaction between PirA^{VP} and APN-1 were generated. Only one out of ten models showed the interaction between PirAB^{VP} and CBR on APN-1 via the interaction between PirA^{VP} and CBR on APN-1. This shows PirA^{VP} is involved in PirAB^{VP} and APN-1 interaction. White arrow indicated the CBR.

FIG. 14—A model of interaction between Cry1a (PDB accession number 1CIY) toxin released by Bacillus thuringiensis and APN-1 protein of Bombyx mori. The model of APN-1 from Bombyx mori was generated by SWISS-model which GMQE and QMEAN values were 0.63 and −3.78, respectively. The model of interaction was generated by using the Gramm-X server (http://vakser.compbio.ku.edu/resources/gramm/grammx/) (Tovchigrechko et al 2006). Domain II and Domain III of Cry toxin bind to the CBR (red-dashed oval) of APN-1.

FIG. 15—A model of interaction between white spot virus capsid protein VP28 (PDB accession number 2ED6) and its receptor Rab7 in Penaeus monodon shrimp. The amino acid sequence of Rab7 was submitted to SWISS-model for modelling which GMQE and QMEAN values were 0.78 and −0.16, respectively. The model of interaction was generated by using the Gramm-X server (http://vakser.compbio.ku.edu/resources/gramm/grammx/) (Tovchigrechko et al 2006).

FIG. 16A-16E—Nucleotide sequences and the predicted amino acid sequences of aminopeptidase N (AN) gene in Penaeus vannamei shrimp. APN isoforms are marked as APN-1-Pv, APN-2-Pv, APN-3-Pv, APN-4-Pv, and APN-5-Pv. In each APN isoforms, the underline residues indicate the transmembrane domain, the box (number 1) indicates the Cry binding region (CBR) and the and boxes indicated the GAMEN (SEQ ID NO: 11) (box number 2) and HEXXHX¹⁸E (SEQ ID NO: 12) motifs (box number 3).

FIG. 17—Predicted models of interaction between PirA^{VP} and APN-1, -2, -3, -4, -5. The blue indicated APN molecules. The red indicated PirA^{VP}. The green indicated Cry binding region (CBR). The interaction model were built by Haddock server (https://wenmr.science.uu.nl/).

FIG. 18—Predicted models of interaction between PirBVP and APN-1, -2, -3, -4, -5. The blue indicated APN molecules. The red indicated PirBVP. The green indicated Cry binding region (CBR). The interaction model were built by Haddock server (https://wenmr.science.uu.nl/).

FIG. 19—Predicted models of interaction between PirABVP and APN-1, -2, -3, -4, -5. The blue indicated APN molecules. The red indicated PirABVP. The green indicated Cry binding region (CBR). The interaction model were built by Haddock server (https://wenmr.science.uu.nl/).

FIG. 20—Interface interaction between PirABVP and APN-1, -2, -3, -4, -5. Pink color indicated PirABVP. Light-blue color indicated APNs. Hot pink and hot blue color indicated interaction between PirABVP and APNs. The interaction interface were built by Prodigy server (https://wenmr.science.uu.nl/prodigy/).

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.10% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. Bodily fluids include, without limitation, amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, fluids and other semi-fluid materials excreted from an organism, cell cultures from bodily fluids, tissue samples, organ samples, shells, exoskeleton, and/or the like. Bodily fluids and/or biological samples may be obtained from an organism, for example by puncture, or other collecting or sampling procedures. Biological samples can be an environmental sample.

The term “environmental sample” refers to samples obtained from the environment such as air, water, soil, animal feed, particulate matter, and/or the like. Environmental samples can be, but are not necessarily, biological samples.

The terms “subject,” “individual,” and “patient” are used interchangeably herein to refer to a vertebrate, preferably a crustacean, and more preferably a shrimp, crayfish, and prawns. Mammals include, but are not limited to, murines, simians, humans, farm animals, sport animals, and pets. Crustaceans include, but are not limited to crabs, lobsters, crayfish, shrimps, prawns, krill, woodlice, and barnacles. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein, “cell type” refers to the more permanent aspects (e.g., a hepatocyte typically can't on its own turn into a neuron) of a cell's identity. Cell state can be thought of as the characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160.

As used herein, “cell state” are used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These time-dependent processes may occur transiently within a stable cell type (as in a transient environmental response), or may lead to a new, distinct type (as in differentiation). Wagner et al., 2016. Nat Biotechnol. 34(111): 1145-1160.

As used herein, “cellular phenotype” refers to the configuration of observable traits in a single cell or a population of cells.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (MW) as opposed to the number-average molecular weight (Mn). Capillary viscometry provides estimates of molecular weight as the inherent viscosity determined from a dilute polymer solution using a particular set of concentration, temperature, and solvent conditions.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein “reduced expression” or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

The term “modification causing said reduced expression” refers to a modification in a gene which affects the expression level of that or another gene such that the expression level of that or another gene is reduced or decreased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “marker” is a term of art and commonly broadly denotes a biological molecule, more particularly an endogenous biological molecule, and/or a detectable portion thereof, whose qualitative and/or quantitative evaluation in a tested object (e.g., in or on a cell, cell population, tissue, organ, or organism, e.g., in a biological sample of a subject) is predictive or informative with respect to one or more aspects of the tested object's phenotype and/or genotype. The terms “marker” and “biomarker” may be used interchangeably throughout this specification.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

The term “modification causing said increased expression” refers to a modification in a gene which affects the expression level of that or another gene such that expression of that or another gene is increased. In particular embodiments, the modification is in a gene relating to an antigen processing pathway. In some embodiments, the modification is in a gene relating to the cross-presentation pathway. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used herein, “identity,” refers to a relationship between two or more nucleotide or polypeptide sequences, as determined by comparing the sequences. In the art, “identity” also refers to the degree of sequence relatedness between polynucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides or polynucleotides of the present disclosure, unless stated otherwise.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, the term “encode” refers to principle that DNA can be transcribed into RNA, which can then be optionally translated into amino acid sequences that can form proteins. Thus, the term encompasses both RNA and protein products produced from DNA through transcription and optional translation.

As used herein, “dose,” “unit dose,” or “dosage” can refer to physically discrete units suitable for use in a subject, each unit containing a predetermined quantity of the one or more Vibrio spp. toxin binding proteins and/or a pharmaceutical or other formulation thereof calculated to produce the desired response or responses in association with its administration.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrastemal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavemous, intracavitary, intracerebral, intracistemal, intracomeal, intracoronal (dental), intracoronary, intracorporus cavemosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating”, and “treatment” can refer generally to obtaining a desired pharmacological and/or physiological effect. The effect can be, but does not necessarily have to be, prophylactic in terms of preventing or partially preventing a disease, symptom or condition thereof, such as a Vibrio spp. infection or disease (e.g. AHPND)]. The effect can be therapeutic in terms of a partial or complete cure of a disease, condition, symptom or adverse effect attributed to the disease, disorder, or condition. The term “treatment” as used herein covers any treatment of a Vibrio spp. infection or disease (e.g. AHPND), in a subject, particularly a non-human organism, more particularly a crustacean, and more particularly a shrimp, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “preventative” and “prevent” refers to hindering or stopping a disease or condition before it occurs, even if undiagnosed, or while the disease or condition is still in the sub-clinical phase.

All publications, published patent documents, and patent applications cited herein are hereby incorporated by reference to the same extent as though each individual publication, published patent document, or patent application was specifically and individually indicated as being incorporated by reference.

### Overview

Acute hepatopancreatic necrosis disease (AHPND) is a lethal disease in marine shrimp that emerged initially in China in 2009 and then spread to other countries in Southeast Asia and the Americas. It is a disease that has caused over $1 billion in losses since its emergence. The etiologic agent was initially identified as Vibrio parahaemolyticus carrying plasmid-borne binary toxin genes, pirA and pirB (Han et al., 2015; Tran et al., 2013). Subsequently other species of Vibrio including V harveyi, V alginolyticus, and more recently Micrococcus luteus have been reported to cause AHPND (Duran-Avelar et al., 2018; Liu et al., 2016). The clinical signs of AHPND include atrophy and discoloration of hepatopancreas, soft shell, gut with discontinuous, and often 100% of mortality occurs in shrimp farms experiencing AHPND outbreak (Han et al., 2015; Lai et al., 2015; Soto-Rodriguez et al., 2014). Histopathology in AHPND animals reveal three different stages of disease development, namely initial, acute and terminal stages. In the initial phase, elongation of epithelial cells in hepatopancreas have been observed, whereas during the acute and terminal phase necrosis of tubular epithelial cells and inflammatory responses characterized by haemocytic infiltration are observed (Soto-Rodriguez et al., 2014). Hepatopancreatic malfunction and secondary bacterial infection are the contributing factors for mortality in infected shrimp (Han et al., 2015).

It is accepted that shrimp protect themselves from non-self objects by innate immunity that encompass humoral and cellular responses (Jiravanichpaisal et al., 2006). Recently, it has been reported that PirA- and PirB-like binary toxin encoded by V parahaemolyticus can be neutralized by either hemocyanin or anti-lipopolysaccharide factor (Boonchuen et al., 2018; Maralit et al., 2018). Although shrimp immune responses to AHPND has been studied in relation to hemocyte and stomach epithelial cells (Boonchuen et al., 2018; Maralit et al., 2018; Soonthornchai et al., 2016), the immune response of shrimp to AHPND has not been elucidated in hepatopancreas that is accepted widely as a target organ of PirA- or PirB-like toxin (Han et al., 2015; Tran et al., 2013).

Currently, there is no effective therapy against AHPND. Antibiotics are not a viable treatment or prevention option as there is a prohibition on the use of antibiotics in farming shrimp. Thus, stocking ponds with Specific Pathogen Free (SPF) post-larvae, biosecurity and pond management remain the only effective strategies to mitigate AHPND epizootics. However, these practices significantly raise the cost of shrimp farming and do not address the ability to control infection if it does present in the population. As such, there exists an unmet need for compositions and techniques to control, treat, and/or prevent AHPND infection in shrimp.

With that said, embodiments disclosed herein can provide proteins, such as native or recombinant proteins and fragments thereof that are capable of binding or otherwise interacting with toxins from Vibrio spp. and their use to treat and/or prevent infection and/or disease or a symptom thereof of a Vibrio spp. infection. Without being bound by theory, it is believed that by binding or otherwise interacting with the Vibrio spp. toxin, the native or recombinant proteins and fragments thereof can be effective to treat and/or prevent infection and/or disease caused by Vibrio spp. and/or their toxins. Also described herein are formulations, such as feed formulations, that contain an amount of one or more the native or recombinant proteins and fragments thereof that are capable of binding or otherwise interacting with toxins from Vibrio spp. Also described herein are methods of treating a Vibrio spp. disease, such as AHPND, by administering a formulation containing an amount of one or more the native or recombinant proteins and fragments thereof that are capable of binding or otherwise interacting with toxins from Vibrio spp. Also described herein are AHPND susceptibility signatures that can be used to identify susceptible and resistant/tolerant organisms. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Vibrio Toxin Binding Proteins

Described herein are embodiments of proteins and fragments thereof that can be capable of binding or otherwise interacting with one or more Vibrio toxins, such as the PirA (e.g., PirA^{VP}), PirB (e.g. PirB^{VP}), and/or PirA- and PirB-like binary toxins. In some embodiments, a Vibrio spp toxin binding protein is APN or a fragment of at least three amino acids thereof, ALP or a fragment of at least three amino acids thereof, or a CAD or a fragment of at least three amino acids thereof. In some embodiments, the Vibrio spp. toxin binding protein is an endogenous host protein (i.e., is a protein native to and endogenous to the host that is or can be infected by the Vibrio spp.). In some embodiments, the Vibrio spp. toxin binding protein is a native exogenous host protein (i.e., a protein that is native to the host species but supplied exogenously to a host). In some embodiments, the Vibrio spp. toxin binding protein is an exogenous protein not native to the host species. In some embodiments, the Vibrio spp. toxin binding protein is an isolated native protein. In some embodiments, the Vibrio spp. toxin binding protein is a recombinant protein. In some embodiments, the recombinant Vibrio toxin binding protein is engineered such that it binds or otherwise interacts with a Vibrio spp. toxin (e.g., PirA (e.g., PirA^{VP}), PirB (e.g. PirB^{VP}), and/or PirA- and PirB-like binary toxins) with a greater affinity and/or specificity than a non-engineered, reference, or native protein. In some embodiments, the recombinant Vibrio toxin binding protein is engineered such that it contains one or more SNPs or other polymorphisms that increase the affinity and/or specificity for a Vibrio toxin (e.g., PirA (e.g., PirA^{VP}), PirB (e.g., PirB^{VP}), and/or PirA- and PirB-like binary toxins) than a non-engineered, reference, or native protein.

In some embodiments, the Vibrio spp. toxin binding protein is a recombinant APN or a fragment thereof, a recombinant ALP or a fragment thereof, or a recombinant CAD or a fragment thereof. In some embodiments, the recombinant APN, ALP, and/or CAD protein is modified relative to a non-engineered, native, or other reference protein such that the recombinant APN, ALP, and/or CAD protein has increased expression, activity, and/or affinity and/or specificity for a Vibrio toxin (e.g., PirA (e.g. PirA^{VP}), PirB (e.g. PirB^{VP}), and/or PirA- and PirB-like binary toxins). In some embodiments, the modification is made in Vibrio toxin binding or interaction region(s) on the Vibrio toxin binding protein. Exemplary regions include the Cry protein binding domain, GAMEN (SEQ ID NO: 11), and HEXXHX¹⁸E (SEQ ID NO: 12) motifs. In some embodiments, the recombinant APN, ALP, and/or CAD protein is/are engineered to contain one or more SNPs or other polymorphisms that increase the affinity and/or specificity for a Vibrio toxin (e.g. PirA (e.g. PirA^{VP}), PirB (e.g. PirB^{VP}), and/or PirA- and PirB-like binary toxins) than a non-engineered, reference, or native protein. In some embodiments, the APN or a fragment thereof can have a sequence that is 50-100% identical to any one of SEQ ID NOS: 2, 4, 6, 8, or 10, such as about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent identical or any value or range of values therein.

In some embodiments, the Vibrio spp. toxin binding protein is a crustacean protein. In some embodiments, the Vibrio spp. toxin binding protein is a shrimp protein.

### Vibrio Toxin Binding Protein Polynucleotides

Also provided herein are polynucleotides and fragments thereof that encode the Vibrio spp. toxin binding protein as described herein. In some embodiments, the Vibrio toxin binding protein or fragment thereof is encoded by a polynucleotide that is about 50-100 percent identical to any one of SEQ ID NOs: 1, 3, 5, 7, or 9 or a fragment thereof, such as about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent identical or any value or range of values therein.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein.

The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid.

The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

### Vectors

Also provided herein are vectors and vector systems containing a Vibrio spp. toxin binding protein encoding polynucleotide. Also provided herein are vectors that can contain one or more of the polynucleotides capable of generating a modified non-human organism described elsewhere herein. The vectors can be useful in producing bacterial, fungal, yeast, plant cells, animal cells, and transgenic animals that can express and/or have modified expression of one or more genes encoding a Vibrio spp toxin binding protein. The vectors can be useful for the in vitro production (cell-based or cell-free production) of Vibrio spp. toxin binding proteins that can be harvested and added to a formulation that can be used to treat or prevent a Vibrio spp. disease or symptom thereof, such as AHPND. Within the scope of this disclosure are vectors containing one or more of the polynucleotide sequences described herein. One or more of the polynucleotides described herein can be included in a vector or vector system. The vectors and/or vector systems can be used, for example, to express one or more of the polynucleotides in a cell, such as a producer cell, to produce viral particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other aspects of the vectors and vector systems are described elsewhere herein.

### Cell-Based Vector Amplification and Expression

Vectors can be designed for expression of one or more polynucleotides described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, crustacean cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2p plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more polynucleotides described herein so as to drive expression of the one or more elements of the polynucleotides described herein described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif (1990) 60-89).

In some embodiments, one or more vectors driving expression of one or more polynucleotides described herein are introduced into a host cell such that expression of the polynucleotide(s) described herein can result in modifying the genome, transcriptome, proteome, and/or epigenome of a non-human organism. For example, different polynucleotides described herein can each be operably linked to separate regulatory elements on separate vectors. RNA(s) of present invention described herein can be delivered to an animal or mammal or cell thereof to produce an animal or mammal or cell thereof that constitutively or inducibly or conditionally expresses different polynucleotides described herein that incorporates one or more polynucleotides described herein or contains one or more cells that incorporates and/or expresses one or more polynucleotides described herein.

In some embodiments, two or more of the elements expressed from the same or different regulatory element(s) can be combined in a single vector, with one or more additional vectors providing any components of the system not included in the first vector. Polynucleotides described herein that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a polynucleotides described herein, embedded within one or more intron sequences (e.g., each in a different intron, two or more in at least one intron, or all in a single intron). In some embodiments, the polynucleotides described herein can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In aspects, the polynucleotides and/or vectors thereof described herein (such as the polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see e.g., Boshart et al., Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the promoter is a shrimp promoter. Exemplary shrimp promoters include, but are not limited to, shrimp beta-actin promoter (see e.g. Shi et al. r Biotechnol (NY). 2016 June;18(3):349-58. doi: 10.1007/s10126-016-9700-1; and hybrid promoters such as any of those described in Puthumana et al., 2016. Cytotechnology. 68:1147-1159.

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments, a constitutive promoter may be employed. Suitable constitutive promoters for mammalian cells are generally known in the art and include, but are not limited to, SV40, CAG, CMV, EF-1a, O-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the polynucleotides described herein described herein are typically placed under control of a plant promoter, i.e., a promoter operable in plant cells. The use of different types of promoters is envisaged. It will be appreciated that for the large-scale production of recombinant proteins, such as those that can be added or included in a formulation (such as those described in greater detail elsewhere herein), crop plants such as tobacco, are attractive for use in the production of recombinant proteins.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the polynucleotides described herein are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the expression of polynucleotides described herein are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more polynucleotides described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., aspects of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-II-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing a polynucleotides described herein to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

One or more of the polynucleotides described herein can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in a polynucleotide described herein such that the selectable marker polypeptide, when translated, is inserted between two amino acids between the N- and C-terminus of the polynucleotides described herein or at the N- and/or C-terminus of the polynucleotides described herein. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the polynucleotides described herein described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more polynucleotides described herein described herein via suitable linker, such as a glycine or glycine serine linkers as short as GS or GG up to (GGGGG)₃ (SEQ ID NO: 13) or (GGGGS)₃ (SEQ ID NO: 14). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the polynucleotides described herein and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated polynucleotides described herein to specific cells, tissues, organs, etc.

### Cell-Free Vector and Polynucleotide Expression

In some embodiments, the polynucleotide encoding one or more polynucleotides described herein can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g. reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding one or more polynucleotides described herein can be codon optimized. In some embodiments, one or more polynucleotides contained in a vector (“vector polynucleotides”) described herein that are in addition to an optionally codon optimized polynucleotide described herein. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.oijp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gown, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal, avian, or crustacean (e.g., a shrimp or cell thereof) as is described elsewhere herein. Such codon optimized sequences are within the ambit of the ordinary skilled artisan in view of the description herein. In some embodiments, the polynucleotide is codon optimized for a specific cell type, such as a stomach cell, digestive gland cell, or gastrointestinal cell of a crustacean, such as a shrimp. In some embodiments, the vector polynucleotide is optimized for expression in a plant cell. In some embodiments, the plant cell is a feed and/or nutrient source for a crustacean or a shrimp.

### Non-Viral Vectors and Carriers

In some embodiments, the vector is a non-viral vector or carrier. In some aspects, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with one or more polynucleotides described herein of the present invention and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as one or more polynucleotides described herein of the present invention.

### Naked Polynucleotides

In some embodiments, one or more polynucleotides described herein can be included in a naked polynucleotide. The term of art “naked polynucleotide” as used herein refers to polynucleotides that are not associated with another molecule (e.g. proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the polynucleotides described herein of the present invention can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g. plasmids and artificial chromosomes), molecules that contain portions that are single stranded and portions that are double stranded (e.g. ribozymes), and the like. In some embodiments, the naked polynucleotide contains only the polynucleotide(s) described herein of the present invention. In some embodiments, the naked polynucleotide can contain other nucleic acids and/or polynucleotides in addition to the polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the polynucleotides described herein of the present invention can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g. minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g. Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In aspects, the S/MAR sequence is located downstream of an actively transcribed polynucleotide (e.g., one or more polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these aspects, the transposon vector lacks one or more polynucleotide sequences encoding proteins required for transposition. In some embodiments, the non-autonomous transposon vectors lack one or more Ac elements.

In some embodiments, a non-viral polynucleotide transposon vector system can include a first polynucleotide vector that contains the polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the polynucleotide(s) of the present invention) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g., one or more of the polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Chemical Carriers

In some embodiments, the polynucleotide(s) of the present invention can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the polynucleotide(s) of the present invention), (2) those capable of targeting specific cells, (3) those capable of increasing delivery of the polynucleotide (such as the polynucleotide(s) of the present invention) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the polynucleotides described herein of the present invention. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticulo endothelial system. In some aspects, the inorganic particles can be optimized to protect an entrapped molecule from degradation, the Suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., superparamagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g. such as the polynucleotide(s) of the present invention). In some embodiments, chemical non-viral carrier systems can include a polynucleotide such as the polynucleotide(s) of the present invention) and a lipid (such as a cationic lipid). These are also referred to in the art as lipoplexes. Other aspects of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the polynucleotide(s) of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g. polymer-based carriers and lipid-based carriers to functionalize these carriers). In some embodiments, the functionalization is targeting a host cell. Suitable polymers that can be included in the polymer-based non-viral carrier can include, but are not limited to, polyethyleneimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the polynucleotide(s) of the present invention), polymethacrylate, and combinations thereof.

In some embodiments, the non-viral carrier can be configured to release a polynucleotide of the present invention that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the polynucleotide(s) of the present invention described herein and an environmental triggering agent response element, and optionally a triggering agent. In some embodiments, the particle can include a polymer that can be selected from the group of polymethacrylates and polyacrylates. In some embodiments, the non-viral particle can include one or more aspects of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the polynucleotide(s) of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as the polynucleotide(s) of the present invention, into a virus particle and producing said virus particle when used alone or with one or more other viral vectors (such as in a viral vector system). Viral vectors and systems thereof can be used for producing viral particles for delivery of and/or expression of one or more polynucleotides of the present invention described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other aspects of viral vectors and viral particles produce therefrom are described elsewhere herein. In some embodiments, the viral vectors are configured to produce replication incompetent viral particles for improved safety of these systems.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors can include those based upon murine leukemia virus (MuLV), gibbon ape leukemia virus (GaLV), Simian immunodeficiency virus (SIV), human immunodeficiency virus (HIV), and combinations thereof (see e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the polynucleotides of the present invention described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in the 3′LTR to create self-inactivating (SIN) vectors having disrupted promoter/enhancer activity of the LTR. In some aspects, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g. promoter(s)) to drive expression of the features present on the packaging vector, and (iii) an “envelope vector” that contains one or more envelope protein genes and upstream promoters. In aspects, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the polynucleotide(s) of the present invention.

In some embodiments, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these aspects, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) (SEQ ID NO: 15) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA (SEQ ID NO: 15) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments, the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In aspects of the helper-dependent adenoviral vector system one vector (the helper) can contain all the viral genes required for replication but contains a conditional gene defect in the packaging domain. The second vector of the system can contain only the ends of the viral genome, one or more polynucleotides of the present invention described herein, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the one or more polynucleotides of the present invention described herein described herein. In some embodiments, the polynucleotide to be delivered via the viral particle produced from a helper-dependent adenoviral vector or system thereof can be up to about 37 kb. Thus, in some embodiments, a adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the polynucleotide(s) of the present invention. In some embodiments, a hybrid-adenoviral vector can include one or more features of a retrovirus and/or an adeno-associated virus. In some embodiments, the hybrid-adenoviral vector can include one or more features of a spuma retrovirus or foamy virus (FV). See e.g. Ehrhardt et al. 2007. Mol. Ther. 15:146-156 and Liu et al. 2007. Mol. Ther. 15:1834-1841, whose techniques and vectors described therein can be modified and adapted for use with the polynucleotide(s) of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use with the polynucleotide(s) of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. Exemplary Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more polynucleotide(s) of the present invention described herein are as used in the foregoing documents, such as WO 2014/093622 (PCT/US2013/074667) and are discussed in greater detail herein.

AAVs are generally known in the art and can be adapted for use with the present embodiments.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g. the inventive polynucleotide(s)), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g. NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered inventive polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the inventive polynucleotide(s) described herein) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., transcripts, proteins, enzymes, mutant forms thereof, fusion proteins thereof, etc.), and virus particles (such as from viral vectors and systems thereof).

One or more polynucleotides of the present invention can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of the polynucleotides of the present invention can be delivered to cells via particles. The term “particle” as used herein refers to any suitable sized particles for delivery of the polynucleotide(s) of the present invention described herein. Suitable sizes include macro-, micro-, and nano-sized particles. In some embodiments, any of the of the polypeptides, polynucleotides, vectors and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in aspects, particle delivery can also be advantageous for other molecules and formulations described elsewhere herein.

### Formulations

Described in several embodiments herein are formulations that can include a protein or fragment thereof capable of binding a Vibrio spp. toxin, such as P PirA (e.g. PirA^{VP}), PirB (e.g. PirB^{VP}), and/or PirA- and PirB-like binary toxins. Exemplary Vibrio toxin binding proteins that can be included in the formulation are described in greater detail elsewhere herein, for example under the heading “VIBRIO TOXIN BINDING PROTEINS” and the Working Examples herein. In some embodiments the formulation incudes APN or a fragment thereof, ALP or a fragment thereof, CAD or a fragment thereof, and any combination thereof. In some embodiments, the formulation includes a modified or recombinant APN or a fragment thereof, a modified or recombinant ALP or a fragment thereof, a modified or recombinant CAD or a fragment thereof, or a combination thereof, where the respective protein was modified such that the binding ability of the protein or fragment thereof to one or more Vibrio toxins (e.g. PirA (e.g. PirA^{VP}), PirB (e.g. PirB^{VP}), and/or PirA- and PirB-like binary toxins) is increased relative to an unmodified or native protein. In some embodiments, the formulation includes a native APN or a fragment thereof, a modified or recombinant ALP or a fragment thereof, or a modified or recombinant CAD or a fragment thereof contains a SNP or other polymorphism that increases the ability of the protein or fragment thereof to bind one or more Vibrio toxins (e.g., PirA (e.g. PirA^{VP}), PirB (e.g. PirB^{VP}), and/or PirA- and PirB-like binary toxins). Such Vibrio binding protein(s) can be considered as active agent(s) within the formulation. In some embodiments, the formulation can be used to treat and/or prevent a disease caused at least in part by Vibrio toxins. In some embodiments, the Vibrio toxin binding protein or fragment thereof includes or is composed entirely of a CRB binding region.

In some embodiments, the formulation can include 1-100 percent (v/v, w/v, or wt. %) of one or more of the Vibrio toxin binding proteins or fragments thereof. In some embodiments, the formulation contains about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5 to/or 100 percent one or more Vibrio toxin binding proteins (v/v, w/v, or wt. %).

The amounts of each of the Vibrio toxin binding proteins that are included in the formulation can range from about 0.001 micrograms to about 1000 grams. The amounts of each of the Vibrio toxin binding proteins that are included in the formulation can range from about 0.001 micrograms to about 0.01 micrograms. The amounts of each of the Vibrio toxin binding proteins that are included in the formulation can range from about 0.01 micrograms to about 0.1 micrograms. The amounts of each of the Vibrio toxin binding proteins that are included in the formulation can range from about 0.1 micrograms to about 1.0 grams. The amounts of each of the Vibrio toxin binding proteins that are included in the formulation can range from about 1.0 grams to about 10 grams. The amounts of each of the Vibrio toxin binding proteins that are included in the formulation can range from about 10 grams to about 100 grams. The amounts of each of the Vibrio toxin binding proteins that are included in the formulation can range from about 100 grams to about 1000 grams.

The amount of each of the one or more Vibrio toxin binding proteins that can be included in the formulation can range from about 0.001 mL to about 1000 L. The amount of each of the one or more Vibrio toxin binding proteins that can be included in the formulation can range from about 0.001 mL to about 0.01 mL. The amount of each of t the one or more Vibrio toxin binding proteins that can be included in the formulation can range from about 0.01 mL to about 0.1 mL. The amount the one or more Vibrio toxin binding proteins that can be included in the formulation can range from about 0.1 mL to about 1.0 L. The amount of each of the one or more Vibrio toxin binding proteins that can be included in the formulation can range from about 1.0 L to about 10 L. The amounts of each of the one or more Vibrio toxin binding proteins that can be included in the formulation can range from about 10 L to about 100 L. The amounts of each of the one or more Vibrio toxin binding proteins that are included in the formulation can range from about 100 L to about 1000 L.

In some embodiments, the amount or effective amount of the one or more of the primary active Vibrio toxin binding protein agent(s) described herein contained in the pharmaceutical formulation can range from about 1 pg/kg to about 10 mg/kg based upon the bodyweight of the subject in need thereof or average bodyweight of the specific subject population to which the formulation can be administered.

### Auxiliary Agents and Other Ingredients

In addition to the primary active agent Vibrio toxin binding protein component, in some embodiments the formulation can optionally contain one or more additional auxiliary (also referred to herein as secondary agents) agents and/or other ingredients (carriers, excipients, colorants, stabilizers and the like). The secondary agents can optionally provide additional therapeutic effects different from, synergistic with, or additive to the primary active agent(s).

In some embodiments, auxiliary active agent can be included in the formulation or can exist as a stand-alone compound or formulation that can be administered contemporaneously or sequentially with the formulations containing one or more Vibrio binding proteins or fragments thereof described elsewhere herein. In embodiments where the auxiliary active agent is a stand-alone compound or formulation, the effective amount of the auxiliary active agent can vary depending on the auxiliary active agent used and can be as described above. The auxiliary active agent can be simultaneously or sequentially administered with the formulations containing one or more Vibrio binding proteins or fragments thereof described above.

The formulation can include a carrier, including but not limited to, a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

In some embodiments, the effective amount of each secondary active agent can each independently range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount can be an effective concentration. In some embodiments, the effective concentration of each secondary active agent can each independently range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In some embodiments, the effective amount of each secondary active agent can each independently range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, each secondary active agent can be included in the formulation at a percentage of the total formulation ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the formulation.

In some embodiments, the amount or effective amount of the one or more of the secondary agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

### Dosage Forms

The formulation that contains a primary active agent Vibrio toxin binding protein component as previously described can be provided in a dosage form. The dosage form can be administered to a subject in need thereof or a population thereof. The dosage form can be effective generate specific concentration, such as an effective concentration, at a given site in the subject in need thereof or in the environment (such as water) of the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a formulation thereof calculated to produce the desired response or responses in association with its administration.

In some embodiments, the Vibrio toxin binding protein component is about 10 to about 100 percent of the total dosage form (v/v, w/v, or wt. %). In some embodiments, the primary active agent Vibrio toxin binding protein component is about 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5 to/or 100 percent of the total dosage form (v/v, w/v, or wt. %). In some embodiments, the primary active agent Vibrio toxin binding protein component is about 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5 to/or 60 percent of the total dosage form (v/v, w/v, or wt. %). In some embodiments, the primary active agent Vibrio toxin binding protein component is about 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5 to/or 50 percent of the total dosage form (v/v, w/v, or wt. %). In some embodiments, the primary active agent Vibrio toxin binding protein component is about 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5 to/or 40 percent of the total dosage form (v/v, w/v, or wt. %). In some embodiments, the primary active agent Vibrio toxin binding protein component is about 20, 20.5, 21, 21.5, 22, 22.5, 23, 23.5, 24, 24.5, 25, 25.5, 26, 26.5, 27, 27.5, 28, 28.5, 29, 29.5 to/or 30 percent of the total dosage form (v/v, w/v, or wt. %).

In some embodiments, the formulation can be a liquid. In some embodiments, the formulation can be a solid. In some embodiments, the formulation can be a substantially dry solid. In some embodiments, the formulation can be formulated as a concentrate that can be diluted for amounts ranges described above. The concentrate can be added to a liquid or a solid. In some embodiments, the solid is a substantially dry solid. In some embodiments, the formulation can be formulated as a feed additive. In some embodiments, the formulation is a feed. In some embodiments, the formulation can be formulated as a water additive. In some embodiments, the formulation can be added to a water source (like or a water habitat (e.g., lake, pond, ocean, artificial pool, artificial pond, tank, and the like).

As previously described the formulations can contain a suitable diluent or carrier. Such diluents or carriers can include, but are not limited to, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active agent(s). In some embodiments, the carrier and/or diluent is an oil, such as a fish oil, or similar oil. In some embodiments, the carrier and/or diluent is DMSO.

The formulations can be sterilized, and optionally, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active compound(s).

The formulations can be provided in various dosage forms that can be administered to a subject or into its environment. Dosage forms of the formulations described herein can be discrete dosage units such as capsules, pellets or tablets, powders or granules, solutions, or suspensions in aqueous or non-aqueous liquids; edible foams or whips, or in oil-in-water liquid emulsions, water-in-oil liquid emulsions, oil-in-water liquid microemulsions, or water-in-oil liquid microemulsions. In some embodiments, the dosage form can also include one or more agents which flavor, preserve, color, or help disperse the formulation. Dosage forms can also be in the form of a liquid solution that can be delivered as a foam, spray, or liquid solution.

Where appropriate, the dosage forms described herein can be microencapsulated. The dosage form can also be prepared to prolong or sustain the release of any ingredient. In some embodiments, one or more of the primary active agent Vibrio toxin binding protein(s) is/are the ingredient(s) whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

For some aspects, the dosage form contains a predetermined amount of a formulation described herein per unit dose. The predetermined amount of the formulation described herein can be an appropriate fraction of the total amount to be administered in a total dose (which can be based on e.g., a time frame (e.g.) minute, hour, day, month, year) or a total amount to treat a disease condition or disorder). Such unit doses may therefore be administered once or more than once a day (e.g., 1, 2, 3, 4, 5, 6, or more times per day). Such unit doses may therefore be administered once or more than once a week (e.g., 1, 2, 3, 4, 5, 6, or more times per week). Such unit doses may therefore be administered once or more than once a week (e.g., 1, 2, 3, 4, 5, 6, or more times per month). Such unit doses may therefore be administered once or more than once a year (e.g., 1, 2, 3, 4, 5, 6, or more times per year). Such formulations may be prepared by any of the methods well known in the art.

In some embodiments, no dosage is given a certain time prior to harvesting the organism for human consumption. This is referred to in the art as “withdrawal time”. In some embodiments, the withdrawal time can range from 0 to 30 days.

Effective dosages and schedules for administering the formulations provided herein may be determined empirically, and making such determinations is within the skill in the art. The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Dosage can vary, and can be administered in one or more dose administrations daily, for one or several days. The range of dosage largely depends on the application of the compositions herein, severity of condition, and its route of administration.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

In some embodiments, the dosage form is adapted for administration through a feed or water source for the subject. In some embodiments, the dosage form is an animal feed, such as a suitable feed for crustaceans. In some embodiments, the dosage form is a feed for shrimp. The feed can include feed forms, which can be pellets, granules, flakes, powder, tablets, paste, or any other suitable feed form. In some embodiments, the feed form is coated with the primary and or optional secondary active ingredient(s). In some embodiments, the primary and or optional secondary active ingredient(s) are dispersed or mixed within the feed forms. Dispersion can be homogenous or heterogenous. The feed can be distributed to the subject for oral consumption to deliver the active agent(s) present in the feed.

In some embodiments, the dosage form, such as a shrimp feed includes, an amount of a flour (e.g., a wheat flour), an amount of soybean meal, and/or an amount of fishmeal. In some embodiments, the amounts of each ingredient of the dosage form are based on an appropriate life stage feed formulation for a crustacean, such as a shrimp. Such feed formulations are generally known in the art.

In some embodiments, the dosage form is adapted for administration via the water environment of the subject. In some embodiments, such a dosage form can include the primary active agent(s) and optional secondary active agent at a concentration or amount such that when administered to the water it is diluted to a concentration or amount that can be effective to treat or prevent a Vibrio spp. infection or disease when ingested or absorbed by subjects within the water. In some embodiments, such dosage forms are solids, powders, gels, foams, or liquids.

### Hepatopancreatic Necrosis Disease Signatures

Described herein are signatures, such as gene signatures, that can be used to detect an organism that is susceptible to or tolerant of AHPND. The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status and/or phenotype of the entire cell (sub)population and/or organism. Furthermore, the signature may be indicative of cells within a population of cells in vivo. The signature may also be used to suggest for instance particular, preventatives, therapies, combination disease control modalities or techniques, and/or to suggest ways to modulate one or more systems within a subject such as an immune or metabolic system. The signatures of the present invention may be discovered by analysis of expression profiles of cell populations and/or of single-cells within a population of cells from isolated samples (e.g., hepatopancreatic samples), thus allowing determination of tolerant or susceptible organisms. The organism susceptibility to AHPND can be determined by specific signatures in cells. The signatures described herein can also be characteristic of a specific cell state or type. The presence of the organism susceptibility to AHPND and any specific cell (sub)types or cell states can be determined by applying the signature genes to bulk sequencing data in a sample.

Also described herein biomarkers (e.g., phenotype specific or cell type) that can be part of a signature and can be used in a method described herein in a variety of diagnostic, preventive, therapeutic, selective breeding and husbandry, and/or population screening and selection, particularly for AHPND susceptible and AHPND tolerant cells and organisms. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein. Specific biomarkers for AHPND tolerant and susceptible cells and/or organisms are described in greater detail elsewhere herein.

Biomarkers are useful in methods of determining AHPND susceptibility and/or tolerance in a subject by detecting a first level of expression, activity and/or function of one or more biomarkers and comparing the detected level or value to a control level or value, wherein a difference in the detected level and the control level indicates that the subject is susceptible to or tolerant of AHPND.

In some embodiments, distinct reference values can represent the prediction of a risk (e.g., an abnormally elevated risk) of being susceptible to or tolerant of AHPND as described elsewhere herein. In some embodiments, distinct reference values can represent predictions of differing degrees of tolerance of or susceptibility to AHPND.

Such comparison(s) can generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of organisms characterised by a particular level of tolerance of or susceptibility to AHPND disease or condition (i.e., for whom said diagnosis, prediction and/or prognosis of the AHPND holds true). Such population may comprise without limitation 2 or more, 10 or more, 100 or more, or even several hundred or more individual organisms or cells.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

In some embodiments, a deviation can refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even 100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March;26(3):317-25). Other suitable detection methods are described elsewhere herein.

Not being bound by a theory, the signatures of the present invention can be microenvironment specific, such as their expression in a particular spatio-temporal context, or in response to exposure to an etiological agent of AHPND. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition and/or susceptibility to AHPND. Not being bound by a theory the presence of specific cells and cell subtypes can be indicative of a particular response to a pathological agent(s) (e.g., an etiological agent(s) of AHPND), such as including increased or decreased susceptibility to the pathological agent(s). The signature can indicate the presence of one particular cell type. In one embodiment, the novel signatures are used to detect multiple cell states or hierarchies that occur in subpopulations of cancer cells that are linked to a particular pathological condition and/or susceptibility to AHPND or linked to a particular outcome or progression of the disease, or linked to a particular response to a prevention and/or treatment of the disease.

The signature according to certain embodiments of the present invention may comprise or consist of one or more genes, proteins and/or epigenetic elements, such as for instance 1, 2, 3, 4, 5, 6, 7, 8, 9, 10, 11, 12 or more. In certain embodiments, the signature may comprise or consist of two or more genes, proteins and/or epigenetic elements, such as for instance 2, 3, 4, 5, 6, 7, 8, 9, 10, 11, 12 or more. In certain embodiments, the signature may comprise or consist of three or more genes, proteins and/or epigenetic elements, such as for instance 3, 4, 5, 6, 7, 8, 9, 10, 11, 12 or more. In certain embodiments, the signature may comprise or consist of four or more genes, proteins and/or epigenetic elements, such as for instance 4, 5, 6, 7, 8, 9, 10, 11, 12 or more. In certain embodiments, the signature may comprise or consist of five or more genes, proteins and/or epigenetic elements, such as for instance 5, 6, 7, 8, 9, 10, 11, 12 or more. In certain embodiments, the signature may comprise or consist of six or more genes, proteins and/or epigenetic elements, such as for instance 6, 7, 8, 9, 10, 11, 12 or more. In certain embodiments, the signature may comprise or consist of seven or more genes, proteins and/or epigenetic elements, such as for instance 7, 8, 9, 10, 11, 12, or more. In certain embodiments, the signature may comprise or consist of eight or more genes, proteins and/or epigenetic elements, such as for instance 8, 9, 10, 11, 12 or more. In certain embodiments, the signature may comprise or consist of nine or more genes, proteins and/or epigenetic elements, such as for instance 9, 10, 11, 12 or more. In certain embodiments, the signature may comprise or consist of twelve or more genes, proteins and/or epigenetic elements, such as for instance 13, 14, 15, or more. It is to be understood that a signature according to the invention may for instance also include genes or proteins as well as epigenetic elements combined.

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing diseased cells or diseased cell (sub)populations, as well as comparing diseased cells or diseased cell (sub)populations with non-diseased cells or non-diseased cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one gene/protein and/or epigenetic element of the signature, such as for instance at least to, at least three, at least four, at least five, at least six, or all genes/proteins and/or epigenetic elements of the signature.

A gene signature can contain one or more genes or gene transcripts (simply transcripts) of interest. A transcript of interest may also be referred to interchangeably as a gene of interest or target sequence. “Target sequence” can refer to any polynucleotide, such as DNA or RNA polynucleotides. In some embodiments, a target sequence is derived from the nucleus or cytoplasm of a cell, and may include nucleic acids in or from mitochondrial, organelles, vesicles, liposomes or particles present within the cell and subjected to a single cell sequencing method, retaining identification of the source cell or subcellular organelle.

A gene of interest may comprise, for example, a mutation, deletion, insertion, translocation, single nucleotide polymorphism (SNP), splice variant or any combination thereof associated with a particular attribute in a gene of interest. In another embodiment, the gene of interest may be a cancer gene. In another embodiment, the gene of interest is a mutated cancer gene, such as a somatic mutation.

Any gene, region or mutation of interest can be used to identify cells containing specific genes, regions or mutations, deletions, insertions, indels, or translocations of interest can be included in the libraries. A gene of interest may be, for example, an AHPND susceptibility gene, which refers to a gene that is differentially expressed between two different cells, organisms, or populations that have or is characteristic of different susceptibility to an agent(s) that is/are capable of causing AHPND. In some embodiments, the AHPND susceptibility gene(s) is/are aminopeptidase-N (APN), alkaline phosphatase (ALP), midgut membrane-bound cadherin (CAD), and combinations thereof. In some embodiments, the signature includes one or more SNPs in one or more of aminopeptidase-N (APN), alkaline phosphatase (ALP), midgut membrane-bound cadherin (CAD), and combinations thereof. In some embodiments, the signature includes one or more SNPs within a Cry Binding Region (CBR), GAMEN (SEQ ID NO: 11) motif, Zn²⁺ binding region(s), and combinations thereof that are present in APN.

Thus, in some embodiments, a signature of AHPND susceptibility can include one or more an AHPND susceptibility gene(s) or gene products, which refers to gene(s) and gene product(s) that is/are differentially expressed between two different cells, organisms, or populations that have or is characteristic of different susceptibility to an agent(s) that is/are capable of causing AHPND. As used herein, “gene product” refers to any product produced from (either directly, such as a transcript, or indirectly such as a protein) a gene. In some embodiments, the AHPND susceptibility gene(s) is/are aminopeptidase-N (APN), alkaline phosphatase (ALP), midgut membrane-bound cadherin (CAD), and combinations thereof.

In some embodiments, the marker gene(s) of interest can include one or more mutations. In some embodiments, the mutations can cause or contribute to any observed differential expression and/or characteristic phenotype of a specific cell type, cell state, and/or organism (e.g., susceptibility to a pathological agent(s) (e.g., those that can result in AHPND). In some instances, the mutation is located anywhere in the gene. The gene of interest can comprise a SNP. The methods described elsewhere herein can be designed to distinguish SNPs within a population and thus can be used to distinguish susceptible strains that differ by a single SNP or detect certain disease and/or AHPND susceptibility specific SNPs, such as but not limited to, disease associated SNPs, such as without limitation AHPND associated SNPs. In some embodiments, the signature includes one or more SNPs in one or more of aminopeptidase-N (APN), alkaline phosphatase (ALP), midgut membrane-bound cadherin (CAD), and combinations thereof. In some embodiments, the signature includes one or more SNPs within a Cry Binding Region (CBR), GAMEN (SEQ ID NO: 11) motif, Zn²⁺ binding region(s), and combinations thereof that are present in APN.

Methods of Detecting Organisms Susceptible and/or Tolerant to AHPND

Generally, the methods described herein can be used to analyze and detect a signature that is characteristic of a cell, cell population, and/or organisms that are susceptible or tolerant to a pathological agent(s), such as the causative agent(s) of AHPND. In some embodiments, the methods described herein can be used to stratify organisms into different populations based on their susceptibility to a pathological agent(s), such as the causative agent(s) of AHPND. Described herein are methods and assays capable of detecting an AHPND susceptibility signature in a cell, cell population, and/or organism. In some embodiments, the organism is a crustacean. In some embodiments, the crustacean is a shrimp, prawn, or crawfish.

In some embodiments, the method can include the step of detecting an AHPND susceptibility signature, such as a signature described elsewhere herein, in a biological sample or a component thereof obtained from a subject or cell(s) thereof. In some embodiments, the subject is a crustacean. In some embodiments, the crustacean is a shrimp, prawn, or crawfish. In some embodiments, the sample is obtained from the hepatopancreas.

In some embodiments, a susceptible subject is detected when the subject has increased or upregulated expression, decreased or downregulated expression, or both of one or more genes of the measured gene signature as compared to a suitable control. In some embodiments, a tolerant subject is detected when the subject has increased or upregulated expression, decreased or downregulated expression, or both of one or more genes of the measured gene signature as compared to a suitable control. A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In some embodiments, the suitable control is a sample prepared from known AHPND susceptible strain of crustacean, such as shrimp, prawn or crawfish. In some embodiments, the suitable control is a sample prepared from known AHPND tolerant or known susceptible strain of crustacean, such as shrimp, prawn or crawfish.

Any suitable techniques of detecting expression of an AHPND susceptibility signature as described herein (e.g., gene, protein, epigenetic, etc. signature) can be used. Non-limiting examples of suitable techniques are described herein.

Suitable techniques include, but are not limited to, an RNA-seq method or technique, an immunoaffinity-based method or technique (e.g. immunohistochemistry, immunocytochemistry, immunoseparation assay, Western analysis, and the like), a polynucleotide sequencing method or technique (e.g. Maxium-Gilbert sequencing, chain-termination sequencing (e.g. Sanger sequencing), shotgun sequencing methods and techniques, bridge PCR, massively parallel signature sequencing, polony sequencing, pyrosequencing, Solexa sequencing, combinatorial probe anchor synthesis, SOLiD sequencing, Ion torrent semiconductor sequencing, nanoball sequencing, heliscope single molecule sequencing, single molecule real time sequencing, nanopore sequencing, microfluidic system-based sequencing, tunneling currents sequencing, sequencing by hybridization, sequencing with mass spectrometry, a RNA polymerase based-sequencing method, an in vitro virus high-throughput method, a bisulfite sequencing technique, or a combination thereof), a PCR based method or technique (e.g. PCR, RT-PCR, qPCR, RT-qPCR, etc.), a protein analysis technique (e.g. mass spectrometry, polypeptide sequencing, an immunoaffinity method or technique, and the like), an epigenome analysis technique, and combinations thereof. Other suitable methods and techniques will be appreciated by those of ordinary skill in the art. In some embodiments, the technique or method may be able to measure the expression at the single-cell level. In some embodiments, the technique may be a single-cell RNA-seq method or technique. Such methods and techniques are generally known in the art and can be adapted for use with the present disclosure.

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)² fragment, a single chain antibody fragment, an Fv fragment, a single chain Fv fragment, a nucleic acid, a lectin, a ligand-binding receptor, affibodies, nanobodies, ankyrins, domain antibodies, alternative antibody scaffolds (e.g., diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

In some embodiments, the method can include or be based on a hybridization assay. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1 SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif (1992).

In certain embodiments, single cell RNA sequencing can be used (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the method can include plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the method can include high-throughput single-cell RNA-seq (see e.g., Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application publication WO2016168584A1; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January;12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the method can include performing single nucleus RNA sequencing to detect and/or measure an amount of a marker described elsewhere herein (see e.g., Swiech et al., 2014, Nature Biotechnology Vol. 33, pp. 102-106; Habib et al., 2016, Science, Vol. 353, Issue 6302, pp. 925-928; Habib et al., 2017, “Massively parallel single-nucleus RNA-seq with DroNc-seq” Nat Methods. 2017 October;14(10):955-958; and International Patent Application Publication WO2017164936, which are herein incorporated by reference in their entirety).

In certain embodiments, the method can include performing involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22;348(6237):910-4. doi: 10.1126/science.aabl601. Epub 2015 May 7; US20160208323A1; US20160060691A1; and WO2017156336A1).

Other suitable assays, methods, and techniques for detecting and/or measuring an amount of a marker described elsewhere herein will be appreciated by one of ordinary skill in the art in view of the description herein.

In some embodiments, differences between AHPND tolerant and AHPND susceptible cells and/or organisms can be determined by using any of the methods described herein. In some embodiments, differences between AHPND tolerant signature and AHPND susceptible signature of cells and/or organisms can be determined by using any of the methods described herein.

In some embodiments, differences between AHPND tolerant and AHPND susceptible cells and/or organisms can determined by a method described herein, which can include comparing a gene expression distribution of cells from a test subject with a gene expression distribution of AHPND tolerant cells, AHPND susceptible cells, AHNPND diseased cells, non-diseased cells, reference, and/or other suitable control as determined by a gene expression, protein expression method and/or or another suitable method described herein.

In some embodiments, the method can include assessing the cell types, subtypes, and/or states present in the sample, which can include analyzing of expression matrices from expression data, performing dimensionality reduction, graph-based clustering and deriving list of cluster-specific genes in order to identify cell types and/or states present in the in vivo system and/or organism. These marker genes may then be used throughout to relate one cell state to another. For example, these marker genes can be used to relate an AHPND susceptible cell (sub)types and/or states to an AHPND tolerant or non-diseased cell (sub(types) and/or states (and vice versa). The same analysis may then be applied to the source material for the sample or a control. From both sets of the expression analysis an initial distribution of gene expression data is obtained. In certain embodiments, the distribution may be a count-based metric for the number of transcripts of each gene present in a cell. Further the clustering and gene expression matrix analysis allow for the identification of key genes in the non-diseased or AHPND tolerant cell-state and the AHPND susceptible cell state, such as differences in the expression of key transcription factors. In certain example embodiments, this may be done conducting differential expression analysis. Other analytic methods can be included or performed on their own. Detection and/or analysis is also described in the Working Examples herein.

In some embodiments, identification of an AHPND tolerant or AHPND susceptible cell or cell population can include detecting a shift, such as a statistically significant shift, in the cell-state as indicated by a modulation (e.g., an increased distance) in the gene expression space between a first cancer cell-state and a second cancer cell state and/or a normal or non-diseased cell. In certain embodiments, the distance is measured by a Euclidean distance, Pearson coefficient, Spearman coefficient, or combination thereof.

In certain embodiments, the gene expression space comprises 5 or more genes, 10 or more genes, 20 or more genes, 30 or more genes, 40 or more genes, 50 or more genes, 100 or more genes, 500 or more genes, or 1000 or more genes. In some embodiments, the gene expression space comprises 12 genes. In certain embodiments, the expression space defines one or more cell pathways. In some embodiments, the expression space is composed of one or more genes selected from APN, ALP, midgut membrane-bound cadherin (CAD), and combinations thereof. In some embodiments, the signature includes one or more SNPs within a Cry Binding Region (CBR), GAMEN (SEQ ID NO: 11) motif, Zn²⁺ binding region(s), and combinations thereof that are present in APN.

The statistically significant shift may be at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%. The statistical shift may include the overall transcriptional identity or the transcriptional identity of one or more genes, gene expression cassettes, or gene expression signatures of the a first cancer cell state compared to a second cancer cell state and/or a normal or non-diseased state (i.e., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of the genes, gene expression cassettes, or gene expression signatures are statistically shifted in a gene expression distribution). A shift of 0% means that there is no difference to the cell states.

A gene distribution may be the average or range of expression of particular genes, gene expression cassettes, or gene expression signatures in an AHPND tolerant cell-state, an AHPND susceptible cell state, and/or a normal or non-diseased cell state (e.g., a plurality of a cells of interest from a subject may be sequenced and a distribution is determined for the expression of genes, gene expression cassettes, or gene expression signatures). In certain embodiments, the distribution is a count-based metric for the number of transcripts of each gene present in a cell. A statistical difference between the distributions indicates a shift. The one or more genes, gene expression cassettes, or gene expression signatures may be selected to compare transcriptional identity based on the one or more genes, gene expression cassettes, or gene expression signatures having the most variance as determined by methods of dimension reduction (e.g., tSNE analysis).

In some embodiments, statistical shifts can be determined by defining a normal or non-diseased cell state, an AHPND tolerant cell state, and/or an AHPND susceptible cell state score. For example, a gene list of key genes enriched in an AHPND tolerant/susceptible model may be defined. To determine the fractional contribution to a cell's transcriptome to that gene list, the total log (scaled UMI+1) expression values for gene with the list of interest are summed and then divided by the total amount of scaled UMI detected in that cell giving a proportion of a cell's transcriptome dedicated to producing those genes. Thus, statistically significant shifts may be shifts in an initial score for the AHPND tolerant cell state score towards the AHPND susceptible cell state score and vice versa.

The term “unique molecular identifiers” (UMI) as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR or multiple displacement amplification (MDA). Unique molecular identifiers can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support. Design and construction of UMIs are generally known in the art and can be used with the methods herein. See e.g., Islam S. et al., 2014. Nature Methods No: 11, 163-166, International Patent Publication No. WO 2014/047561. Other barcoding and tagging methods can be used with the invention herein, which are also known in the art. See e.g. Kress et al., “Use of DNA barcodes to identify flowering plants” Proc. Natl. Acad. Sci. U.S.A. 102(23):8369-8374 (2005), Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009), CBOL Plant Working Group, “A DNA barcode for land plants” PNAS 106(31):12794-12797 (2009), Kress et al., “DNA barcodes: Genes, genomics, and bioinformatics” PNAS 105(8):2761-2762 (2008), Lahaye et al., “DNA barcoding the floras of biodiversity hotspots” Proc Natl Acad Sci USA 105(8):2923-2928 (2008), Ausubel, J., “A botanical macroscope” Proceedings of the National Academy of Sciences 106(31):12569 (2009), Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17;106(7):2289-94).

In some embodiments, the method can include generating a sequencing library. Methods of generating such a library are generally known in the art and can be used with the invention described herein.

Other methods for assessing differences in the AHPND tolerant cells/organisms and AHPND susceptible cells/organisms can be employed. In certain example embodiments, an assessment of differences in the AHPND tolerant and the AHPND susceptible proteome may be used to further identify key differences in cell type and sub-types or cells. states. For example, isobaric mass tag labeling and liquid chromatography mass spectroscopy may be used to determine relative protein abundances in the ex vivo and in vivo systems. Description provided elsewhere herein further disclosure on leveraging proteome analysis within the context of the methods disclosed herein.

The methods described herein can be used to identify and isolate cells and/or non-human organisms that are tolerant and/or susceptible to AHPND. The identified non-human organisms, such as crustaceans, can be used to identify lineages that can be used to improve the tolerance of an organism to AHPND. For example, lineages of non-human organisms, such as crustaceans, tolerant can be used in breeding programs to raise generations of non-human organisms that can have improved AHPND tolerance expression signatures, which can be used in animal production, such as aquaculture. In some embodiments, the identified non-human organisms can be used in aquaculture production. In some embodiments, the identified non-human organisms can be used in as parents in a breeding program for aquaculture production.

Other suitable assays, methods, and techniques for detecting and/or measuring an amount of a marker described elsewhere herein will be appreciated by one of ordinary skill in the art in view of the description herein.

Cells and Organisms with Tolerance to Ahpnd

Also described herein are organisms and cells, particularly non-human organisms, such as crustaceans, and cells that have an AHPND susceptibility signature that indicates that the organism or cell is tolerant/resistant to the Vibrio spp., particularly the toxins that are causative of AHPND. Such signatures are described in greater detail elsewhere herein. In some embodiments, such organisms and/or cells can be generated by a selective mating program. In some embodiments, such organisms and/or cells can be generated by a suitable genetic modification technique. In some embodiments, the organisms and/or cells are modified such that they have an AHPND susceptibility signature that indicates that the organism or cell is tolerant/resistant to the Vibrio spp., particularly the toxins that are causative of AHPND. Such signatures are described in greater detail elsewhere herein.

In some embodiments, the modified non-human organism is a non-human organism that can be infected by the causative agent(s) of AHPND. In some embodiments, the modified organism is a crustacean. In some embodiments, the modified non-human organism is a shrimp.

Methods and techniques of genetically modifying crustaceans, such as shrimp, are generally known in the art (see e.g., Beardmore and Porter. 2002. FAO Fisheries Circular no. 989; Yang et al. 2016. March Drugs. 14(8) pii: E152. doi: 10.3390/md14080152; Chen at al. 2019. March Biotechnol. February;21(1):9-18. doi: 10.1007/s10126-018-9862-0; Li and Tsai. 2000. Mol. Reprod Dev. 56(2):149-154; Zhang et al., 2018. Fish Shellfish Immunol. 77:244-251. doi: 10.1016/j.fsi.2018.04.002; Gui et al. 2016. Nov. 8;6(11):3757-3764. doi: 10.1534/g3.116.034082, the teachings of which can be adapted for use in the present invention.

Methods of genetic modification can include, but are not limited to, traditional knock in and knock down approaches based on homologous recombination, as well as more recent nuclease-based approaches such as zinc-finger nucleases, transcription activator-like effector nucleases, and CRISPR-Cas systems. Such systems and techniques are known in the art and can be adapted for use with the present invention.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g. Shmakov et al. (2015) “Discovery and Functional Characterization of Diverse Class 2 CRISPR-Cas Systems”, Molecular Cell, DOI: dx.doi.org/10.1016/j.molcel.2015.10.008.

In some embodiments, a meganuclease or system thereof can be used to modify a polynucleotide and produce a modified cell or organism. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

In some embodiments, a TALEN or system thereof can be used to modify a polynucleotide and produce a modified cell or organism. The structure and function of TALENs is further described in, for example, Moscou et al., Science 326:1501 (2009); Boch et al., Science 326:1509-1512 (2009); and Zhang et al., Nature Biotechnology 29:149-153 (2011).

In some embodiments, the polynucleotide is modified using a Zinc Finger nuclease or system thereof and can be used to generate a modified cell or organism described herein. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

Other genetic modification systems that can be used to modify cells as described herein include, but are not limited to, meganucleases, transposon/transposase systems, RNAi, CRISPRi, Prime editing systems, CRISPR-transposase systems, and/or the like.

Treating and/or Preventing Ahpnd

Described herein are methods of treating and/or preventing AHPND infection in a subject organism, such as a non-human organism, preferably a crustacean (including but not limited to a shrimp). In some embodiments, the method can include feeding a feed formulation that includes one or more proteins or fragments thereof capable of binding a Vibrio spp. toxin, such as PirA (e.g. PirA^{VP}), PirB (e.g. PirB^{VP}), and/or PirA- and PirB-like binary toxins. When present in the feed, which can be added to the organism environment, such proteins and fragments thereof can bind a Vibrio spp. toxin when present in the environment or present in the gastrointestinal tract of an organism that has consumed the feed. In this way, the bound toxin is effectively neutralized thereby reducing the severity of or eliminating a resulting disease in the organism.

The formulations described herein can be administered to a subject in need thereof via any appropriate route. In some embodiments, the method can include administering an amount of a formulation as described elsewhere herein to a subject. The method can include contacting a Vibrio spp. with an amount of a formulation described herein. The method can include administering the formulation to the environment of the subject such that the subject can ingest or absorb the formulation or a component thereof. The subject in need thereof can be a non-human animal. In some embodiments, the subject can be a crustacean. In some embodiments, subject is a shrimp. In some embodiments, the formulation can be delivered to the subject through its feed and/or water. In some embodiments, such as for use in aquaculture, the formulation can be delivered through the water that is the subject's habitat or environment. Such water sources include natural ponds, lakes, oceans, rivers streams, and the like. Such water sources can include artificial habitats such as tanks, pools, ponds, and the like.

As previously discussed, the formulation can be effective to treat or prevent an infection caused by a Vibrio spp in a subject to which it is delivered. In some embodiments, the formulation can be effective to reduce, inhibit, and/or eliminate pathogenicity of a Vibrio spp., such as in a subject or environment to which it is delivered. In some embodiments, the formulation can be effective to reduce, inhibit, and/or eliminate growth and/or development of a Vibrio spp., such as in a subject or environment to which it is delivered. In some embodiments, the formulation can be effective to kill a Vibrio spp., such as in a subject or environment to which it is delivered. In some embodiments, the subject has or is suspected of being infected with an organism of a Vibrio spp. In some embodiments, the subject is exposed to an organism of a Vibrio spp.

Also described herein are methods of treating or preventing a Vibrio spp., infection in a subject comprising: administering amount of a formulation as described herein to a subject. The subject can be a non-human animal. The subject can be a crustacean. The crustacean is a shrimp.

In some embodiments, the method can include reducing, inhibiting, and/or eliminating pathogenicity of a Vibrio spp., such as in a subject or environment to which a formulation described herein is delivered. In some embodiments, the method can include reducing, inhibiting, and/or eliminating growth and/or development of a Vibrio spp., such as in a subject or environment to which a formulation described herein is delivered. In some embodiments, the method can include killing a Vibrio spp., such as in a subject or environment to which a formulation described herein is delivered.

The formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more times hourly, daily, monthly, or yearly). In some embodiments, the formulations or dosage forms thereof described herein can be administered continuously over a period of time ranging from minutes to hours to days. Devices and dosages forms are known in the art and described herein that are effective to provide continuous administration of the formulations described herein. In some embodiments, the first one or a few initial amount(s) administered can be a higher dose than subsequent doses. This is typically referred to in the art as a loading dose or doses and a maintenance dose, respectively. In some embodiments, the formulations can be administered such that the doses over time are tapered (increased or decreased) overtime so as to wean a subject gradually off of a formulation or gradually introduce a subject to the formulation.

Also described herein are methods of treating AHPND infection that can include detecting AHPND tolerant and/or susceptible non-human organisms, such as crustaceans, in a population thereof and administering a suitable treatment and/or preventative based on detection of AHPND tolerant and/or susceptible non-human organisms. In some embodiments, detecting AHPND tolerant and/or susceptible non-human organisms include detecting and/and or measuring a change in phenotype, characteristic, signature, and/or activity, function and/or expression of one or more of the biomarkers selected from APN, ALP, CAD and combinations thereof; SNPs in one or more of APN, ALP, CAD or a combination thereof. In some embodiments, the signature includes one or more SNPs in one or more of aminopeptidase-N (APN), alkaline phosphatase (ALP), midgut membrane-bound cadherin (CAD), and combinations thereof. In some embodiments, the signature includes one or more SNPs within a Cry Binding Region (CBR), GAMEN (SEQ ID NO: 11) motif, Zn²⁺ binding region(s), and combinations thereof that are present in APN.

Suitable treatments and/or preventatives can include, but are not limited to, to those compositions, methods, and techniques currently used to manage, mitigate, control, or treat AHPND. It will be appreciated that the signatures and methods described herein can allow for a different and more precise timing and/or amount of a treatment/prevention to be applied, even if such methods are currently used. In some embodiments the treatment and/or prevention is or includes feeding a subject organism with a feed formulation described elsewhere herein that includes one or more proteins or fragments thereof capable of binding a Vibrio spp. toxin, such as PirA (e.g. PirA^{VP}), PirB (e.g. PirB^{VP}), and/or PirA- and PirB-like binary toxins. Exemplary feed formulations are described in greater detail elsewhere herein. In other words, the signatures and methods described herein can change how currently treatments and/or preventions are used and the methods by which they are used. The methods and signatures described herein can, in some embodiments, result in less and/or more efficient use of treatments and preventatives, which can lead to reduced toxicity, waste, and/or costs.

### Kits

The formulations described herein and/or pharmaceutical formulations thereof described herein can be presented as a combination kit. As used herein, the terms “combination kit” or “kit of parts” refers to the compounds, or pharmaceutical formulations and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g., active agents) contained in the kit are administered simultaneously, the combination kit can contain the active agents in a single pharmaceutical formulation (e.g., a tablet) or in separate pharmaceutical formulations. In some embodiments, the kit contains one or more agents or reagents for carrying out a method of detecting an AHPND susceptibility signature in an organism or a sample therefrom.

When the agents are not administered simultaneously, the combination kit can contain each agent in separate pharmaceutical formulations. The separate pharmaceutical formulations can be contained in a single package or in separate packages within the kit.

The combination kit can also include instructions printed on or otherwise contained in a tangible medium of expression. The instructions can provide information regarding the content of the compound or pharmaceutical formulations contained therein, safety information regarding the content of the compound(s) or pharmaceutical formulation(s) contained therein, information regarding the dosages, indications for use, and/or recommended treatment regimen(s) for the compound(s) and/or pharmaceutical formulations contained therein. The instructions can provide directions for administering the compounds, compositions, pharmaceutical formulations, or salts thereof to a subject having, suspected of having, or predisposed to a disease, disorder, or condition described elsewhere herein. The instructions can provide directions for administering the compounds, compositions, pharmaceutical formulations, or salts thereof to a subject having, suspected of having, or predisposed to developing Vibrio spp. infection or a symptom thereof formulations and/or co-treatments described herein that can be included in the kit. The instructions can provide for determining an AHPND susceptibility signature described elsewhere herein.

Further embodiments are illustrated in the following Examples which are given for illustrative purposes only and are not intended to limit the scope of the invention.

### Sequences

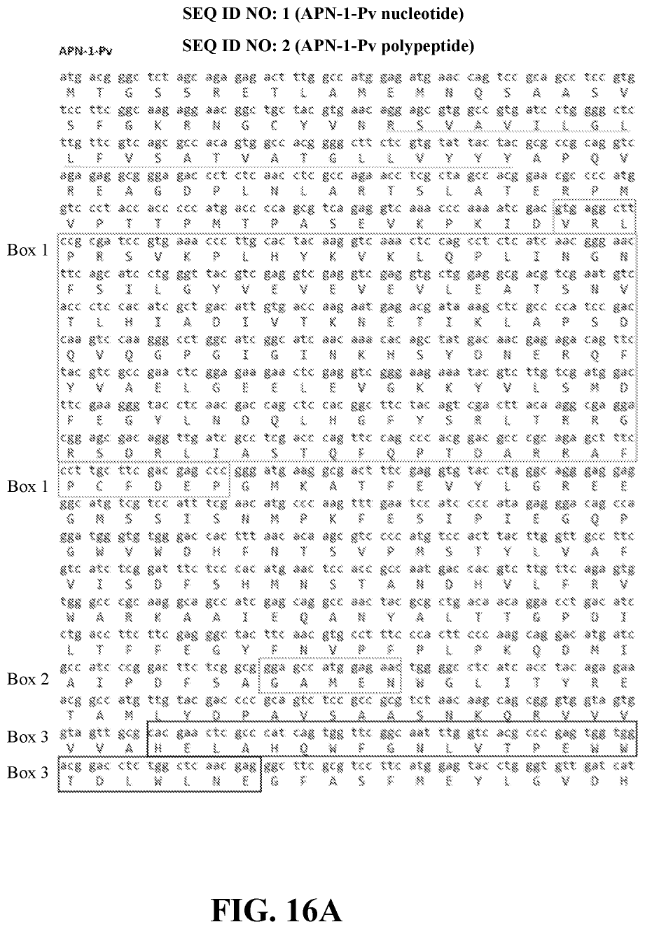

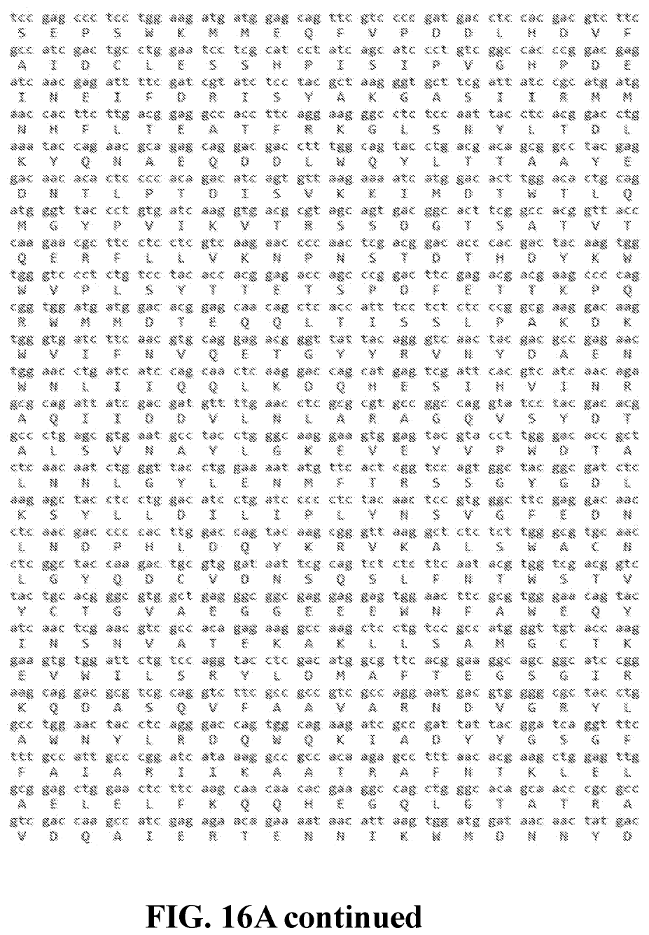

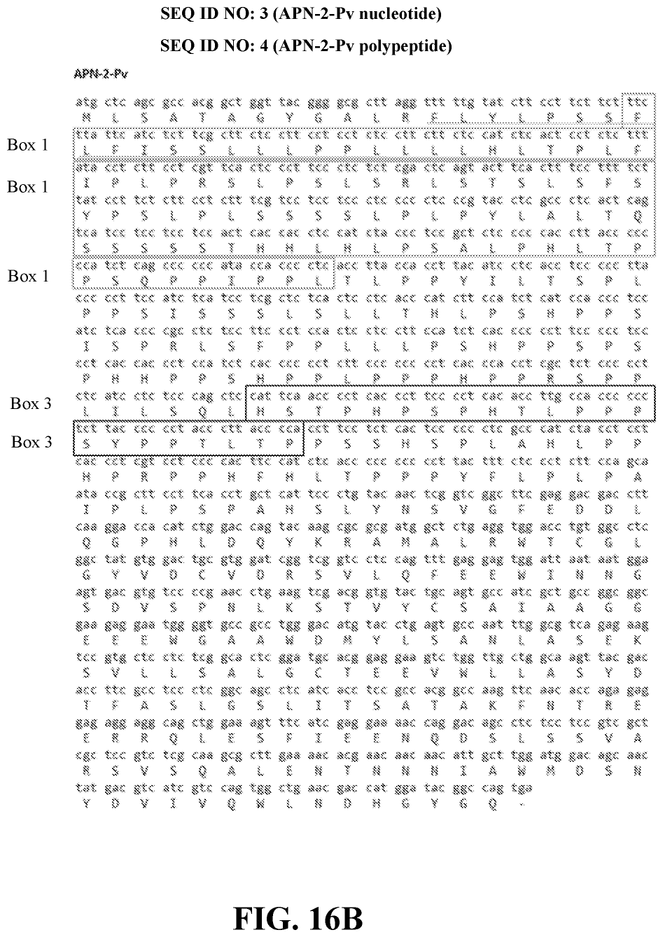

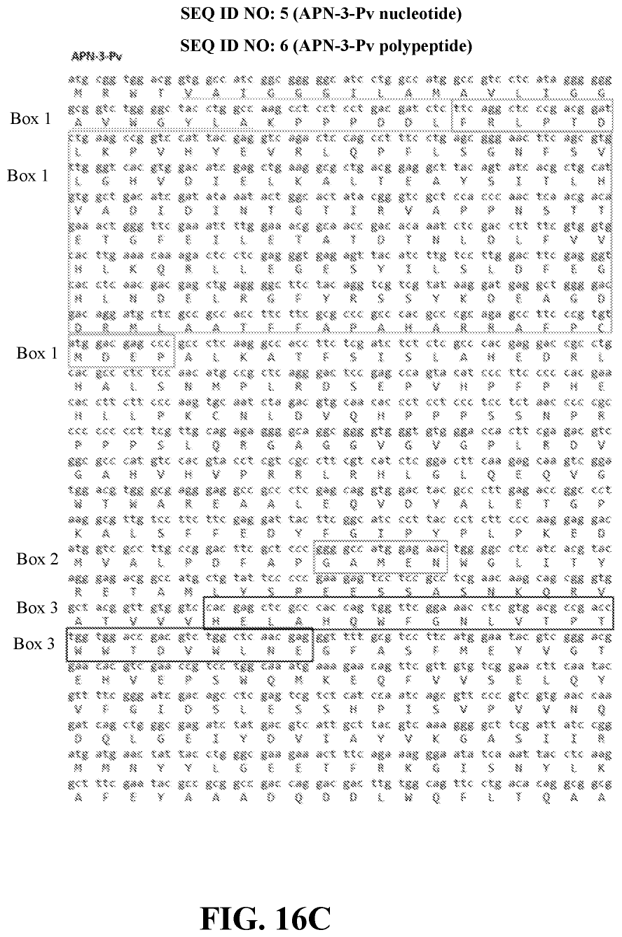

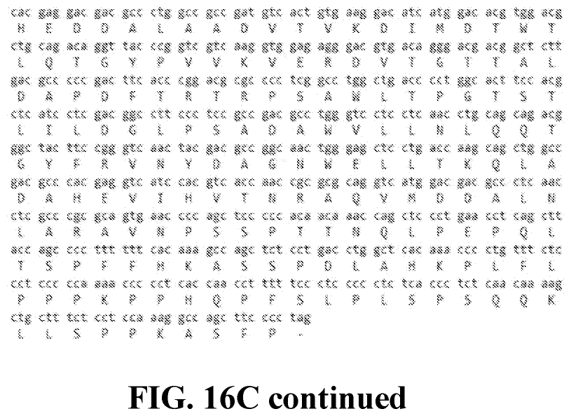

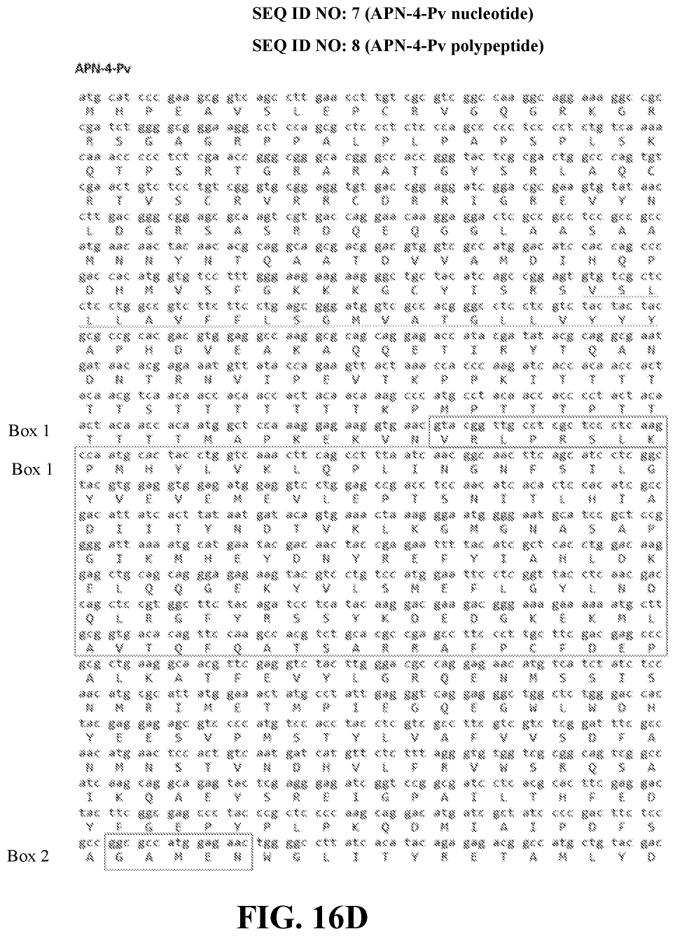

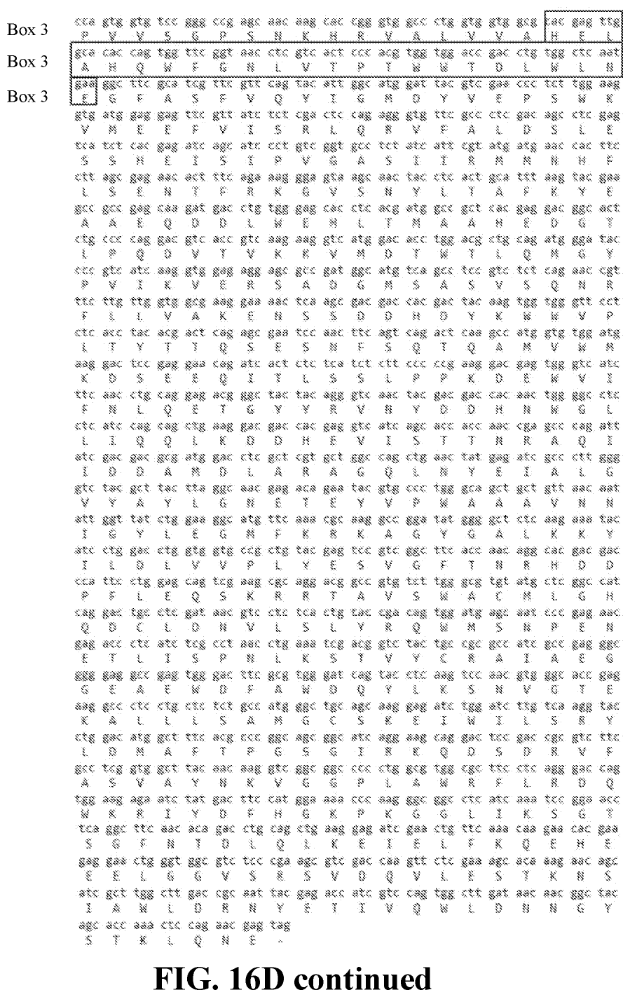

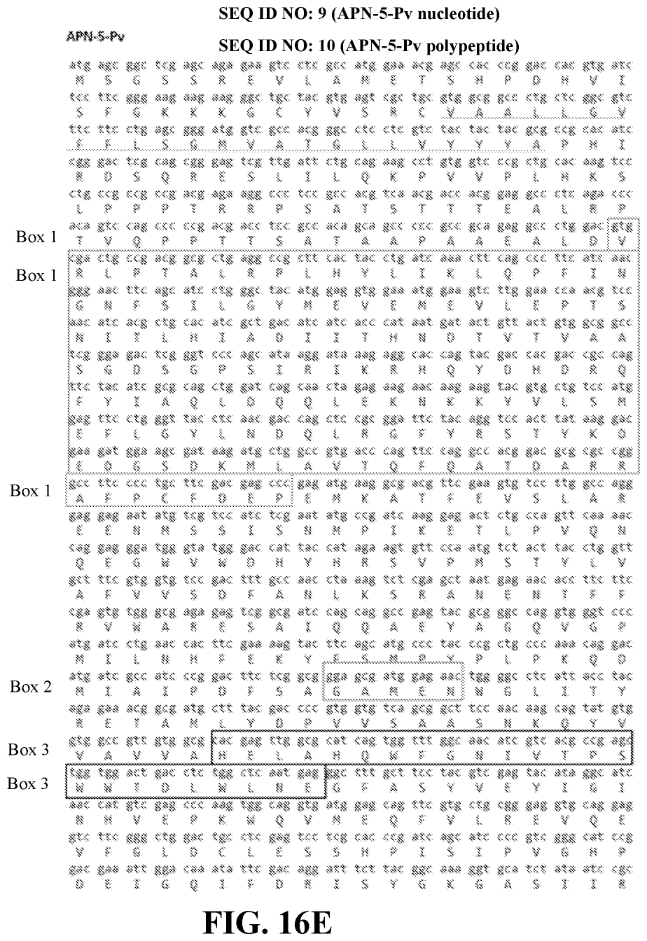

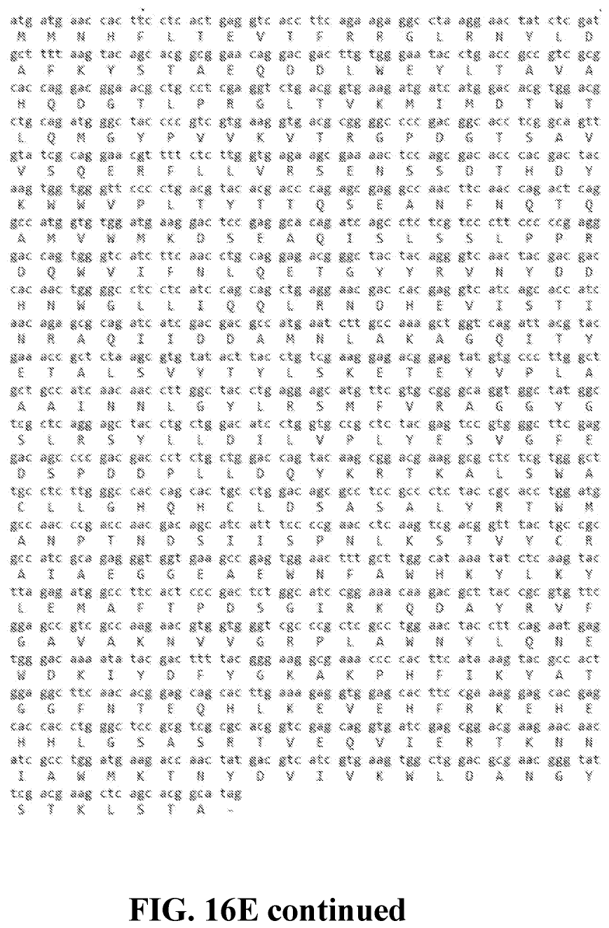

FIGS. 16A-16E show the nucleotide sequences and the predicted amino acid sequences of aminopeptidase N (AN) gene in Penaeus vannamei shrimp (SEQ ID NOS: 1-10) APN isoforms are marked as APN-1-Pv, APN-2-Pv, APN-3-Pv, APN-4-Pv, and APN-5-Pv. In each APN isoforms, the underline residues indicate the transmembrane domain, the red box (number 1) indicates the Cry binding region (CBR) and the Green and black boxes indicated the GAMEN (SEQ ID NO: 11) (box number 2) and HEXXHX¹⁸E (SEQ ID NO: 12) motifs (box number 3).

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Acute hepatopancreatic necrosis disease (AHPND) is a bacterial disease causing serious economic losses to shrimp industry globally since it was reported in 2009. The causative agents of AHPND are Vibrio spp. carrying plasmids containing the pirA and pirB genes which encode binary toxins, PirABVP. The binary toxin PirABVP has a tertiary structure similar to “Cry” protein produced by a bacterium, Bacillus thuringiensis. The Cry protein has insecticidal properties against many insect species belonging to the orders Lepidoptera, Diptera, Coleoptera and Hymenoptera. The Cry toxin binds to three cell receptors including Aminopeptidase-N (APN), Alkaline phosphatase (ALP) and midgut membrane-bound cadherin (CAD).

As is demonstrated in at least this Example, homologs of APN, ALP and CAD were identified in shrimp. Additionally, in silico analysis revealed that the Cry Binding Region (CBR), GAMEN (SEQ ID NO: 11) motif and Zn²⁺ binding regions are present in APN protein in shrimp. Virtual Screening (VS) results showed that PirAVP, PirBVP and PirABVP interacts to APN but only PirAVP binds to CBR region of APN molecule. This shows that APN is a receptor for PirABVP. Recombinant APN, ALP and CAD proteins can be added to shrimp feed to produce functional feed to block PirABVP and protect shrimp from AHPND. Moreover, gene SNPs and/or gene expression of these receptors can be used as markers to differentiate AHPND resistant genetic lines of shrimp from AHPND susceptible shrimp line. Finally, single nucleotide polymorphisms (SNPs) in functional domains involved in PirABVP binding can be used as marker for developing AHPND resistant lines of shrimp in captive breeding programs.

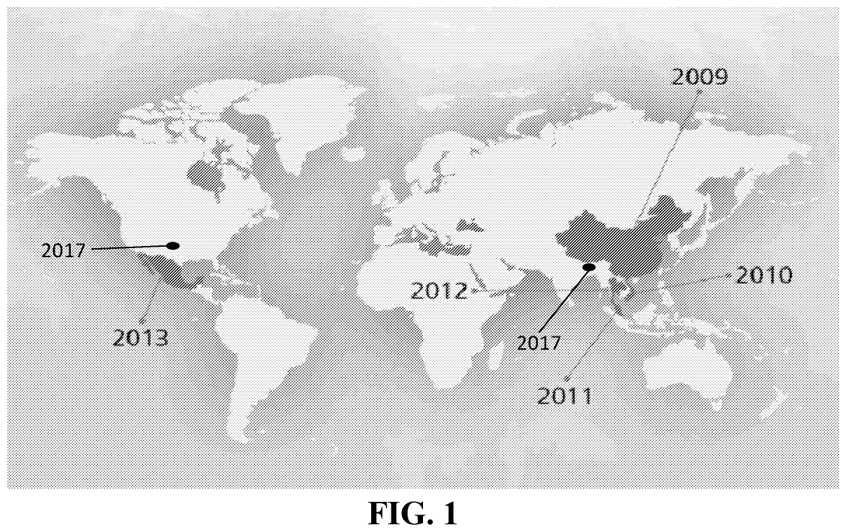

FIG. 1 shows the distribution of AHPND in the world (modified from Zorriehzahra and Banaederakhshan, 2015). The red color indicated the presence of AHPND. AHPND was first reported in China in 2009. Since then, it was reported in several shrimp producing countries including Vietnam (2010), Malaysia (2011), Thailand (2012), Mexico (2013), US (2017), Bangladesh (2017).

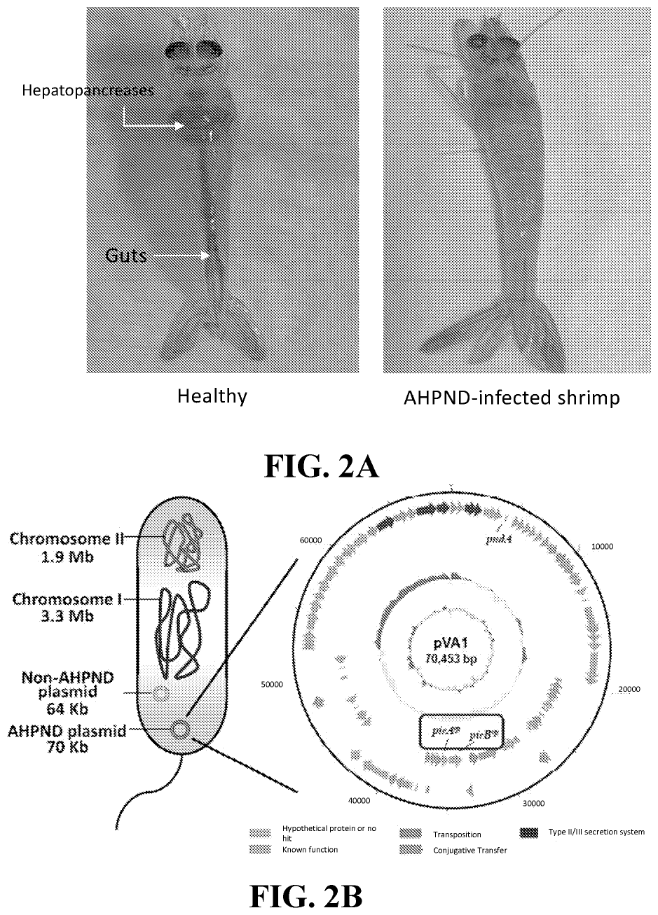

FIGS. 2A-2B show clinical signs and etiological agent of acute hepatopancreatic necrosis disease (AHPND). (FIG. 2A) Clinical sign of AHPND in Penaeus vannamei. The animal on the left represents a healthy animal and the animal on the right represents a AHPND affected animal. The hepatopancreas in the healthy animal is dark brown and the gut is full whereas in diseased animal, the hepatopancreas is pale and the gut is empty (Taken from Kaneshamoorthy et al., 2020). (FIG. 2B) A schematic representation of Vibrio parahaemolyticus cell (left) displaying genomic content, two chromosomes and the plasmid DNAs. A genetic map of the 70.453 kb plasmid DNA containing the binary toxin genes, pirA and pirB is shown on the right (Taken from Lee et al., 2014).

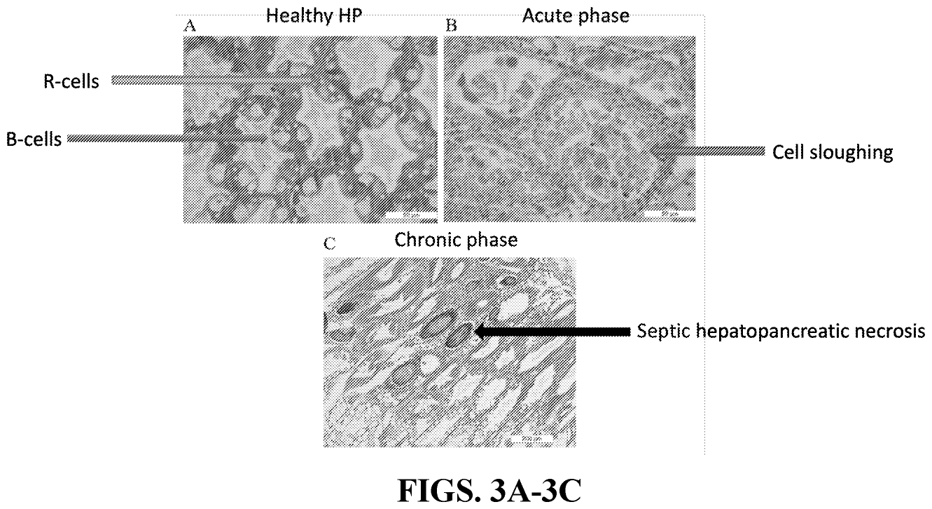

FIGS. 3A-3C show histopathology of hepatopancreases (HP) collected from AHPND-infected shrimp and healthy shrimp. (FIG. 3A) The HP collected from healthy shrimp showed normal structure of tubule and epithelial cells in the hepatopancrease including R-cells (arrow) and B-cells (FIG. 3B) (arrow). Hepatopancreas tissue section from AHPND infected animal displaying acute phase infection characterized by the atrophy of hepatopancreatic tubule, depletion of B- and R-cells, and sloughing of HP tubule epithelial cells and remnants of necrotic cells in the lumen of the HP tubule (arrow) (FIG. 3C) the AHPND-infected HP displaying the chronic phase infection which resemble to septic hepatopancreatic necrosis (SHPN) (arrow) showed only a few tubules with epithelial necrosis accompanied by bacteria and inflammation.

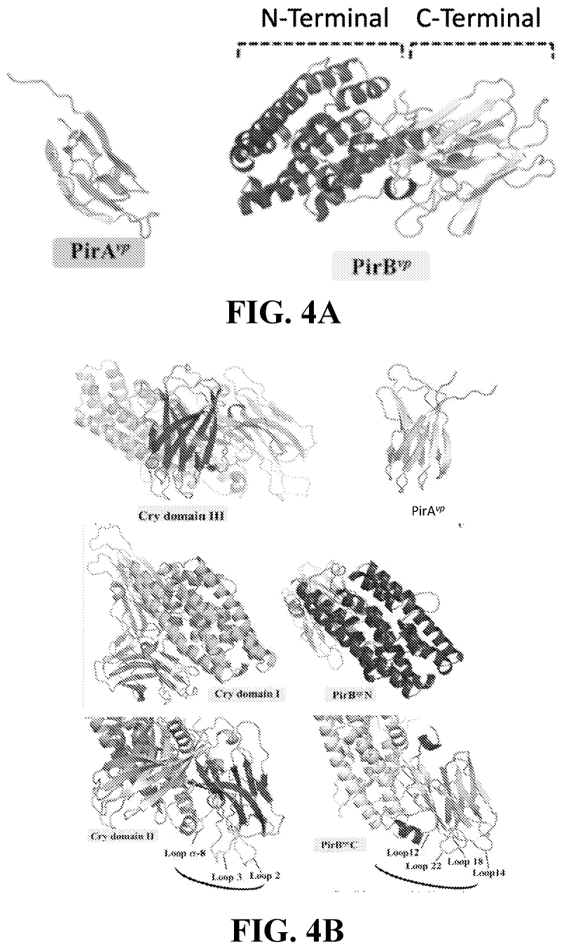

FIGS. 4A-4B show a comparison of PirAB^{VP} structure with structure of Cry toxin released by Bacillus thuringiensis. (FIG. 4A) The protein structure of PirA^{VP} and PirB^{VP}(Taken from Lin et al., 2017). The PirA^{VP} has beta sheet in structure. PirB^{VP} contains alpha-helix in N-terminal and Beta sheet in C-terminal in structure. (FIG. 4B) The similarity in structure between PirAB^{VP} and Cry toxin. The PirA shares similar structure with Cry domain III. The PirB^{VP} N-terminal shares similar structure with Cry domain I and PirB^{VP} C-terminal structure shares similarity to Cry domain II. The shared structure of PirAB^{VP} and Cry toxin suggested that the mode action of PirAB in shrimp might be similar to mode action of Cry toxin in insects.

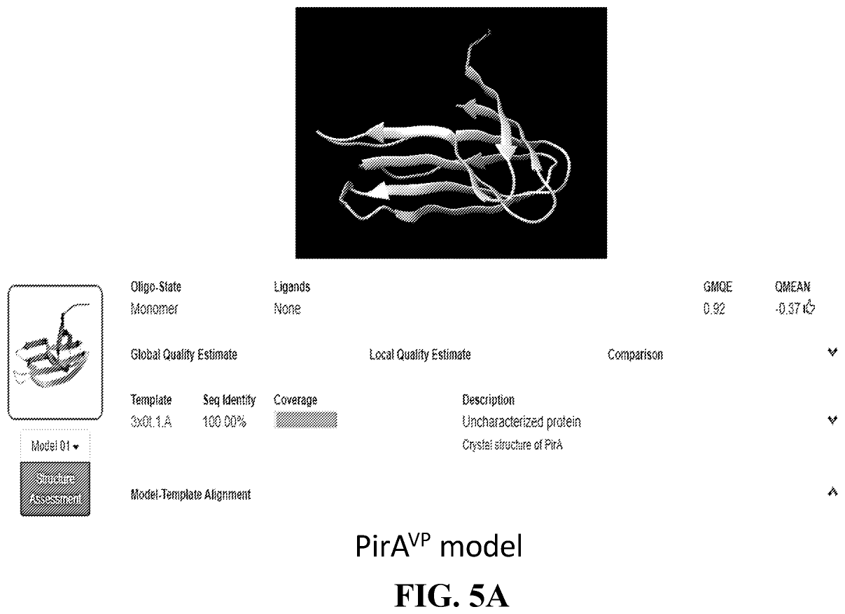

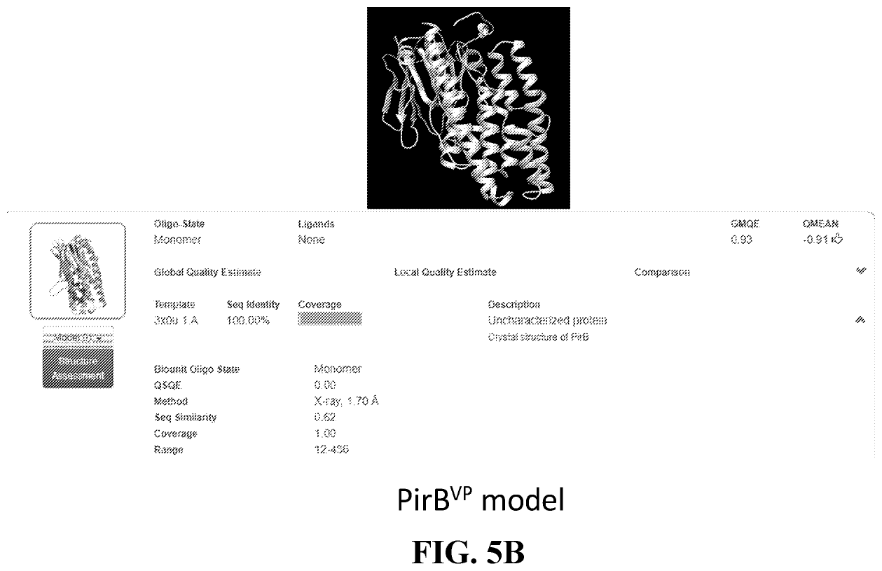

FIGS. 5A-5B show a predicted structure of PirA^{VP}/PirB^{VP} toxin of Vibrio parahaemolyticus causing AHPND (FIG. 5A) The structure of PirA^{VP} were built using Swiss-model (https://swissmodel.expasy.org/interactive). (FIG. 5B) The structure of PirB^{VP} were built using Swiss-model (https://swissmodel.expasy.org/interactive). The Global Model Quality Estimation (GMQE) values of proposed PirA models ranged from 0.92-0.93. The Qualitative model energy analysis (QMEAN) value of the proposed PirA/PirB^{VP} models ranged from −0.37 to −0.91. The QMEAN and GMQE values are used as a criteria to determine the quality of a predicted tertiary structure. The GMQE value ranges from 0 to 1.0. A GMQE value close to 1.0 indicates the high quality of a model. A QMEAN value close to 0 indicates the proposed model is reliable. The “thumb up” sign in QMEAN indicates an acceptable value.

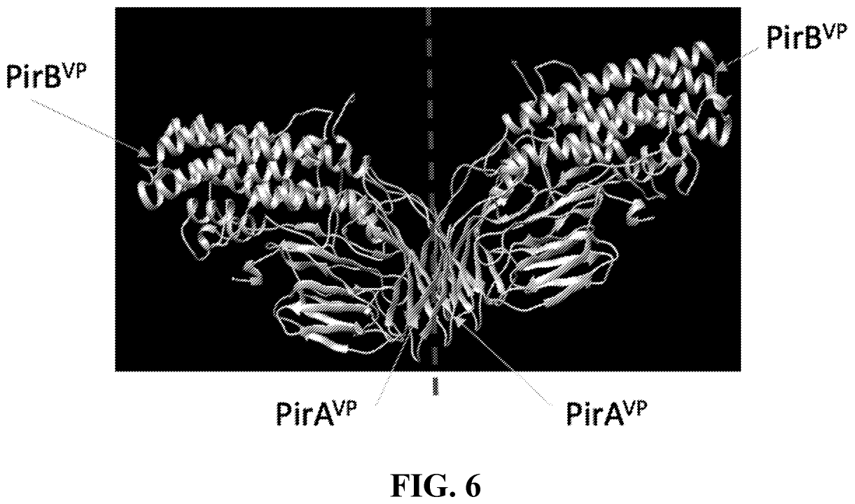

FIG. 6 shows a predicted PirAB^{VP} structure was generated using the Gramm-X server (http://vakser.compbio.ku.edu/resources/gramm/grammx/) (Tovchigrechko et al 2006). The binary toxin contains four subunits, two of PirA and two of PirB. The interaction interface (dotted line) between two heterodimer (PirA^{VP}/PirB^{VP}) occurred between two PirA molecules. Arrows indicated the PirB^{VP}, arrows indicated PirAB^{VP}. First, the monomer PirA^{VP} and PirB^{VP} models were built using SWISS-model. Then, a heterodimers of PirA^{VP} and PirB^{VP} models were built using Gramm-X server (http://vakser.compbio.ku.edu/resources/gramm/grammx/) (Tovchigrechko et al 2006). Then, a tetraheteromer between two heterodimers of PirA^{VP} and PirB^{vp} was generated using the Gramm-X server. (http://vakser.compbio.ku.edu/resources/gramm/grammx/) (Tovchigrechko et al 2006).

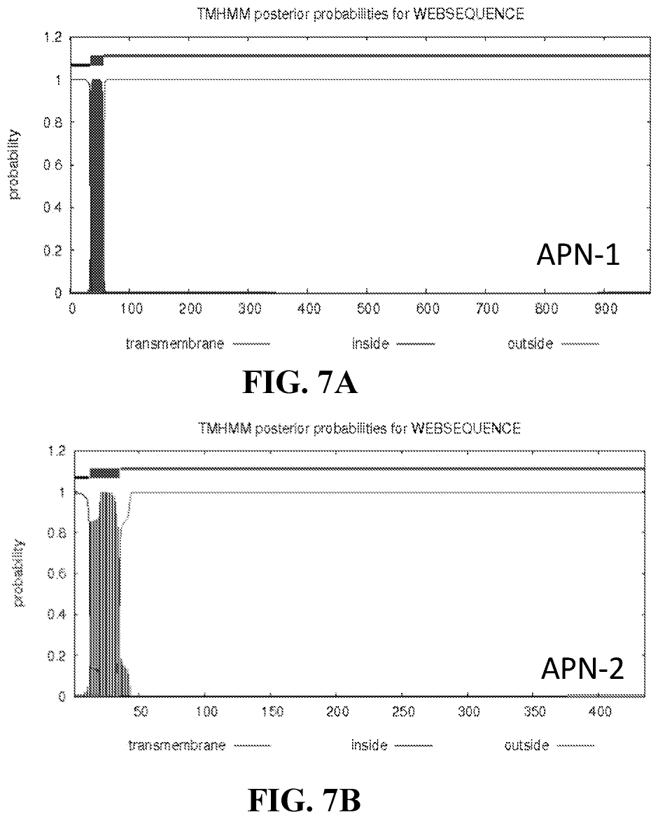

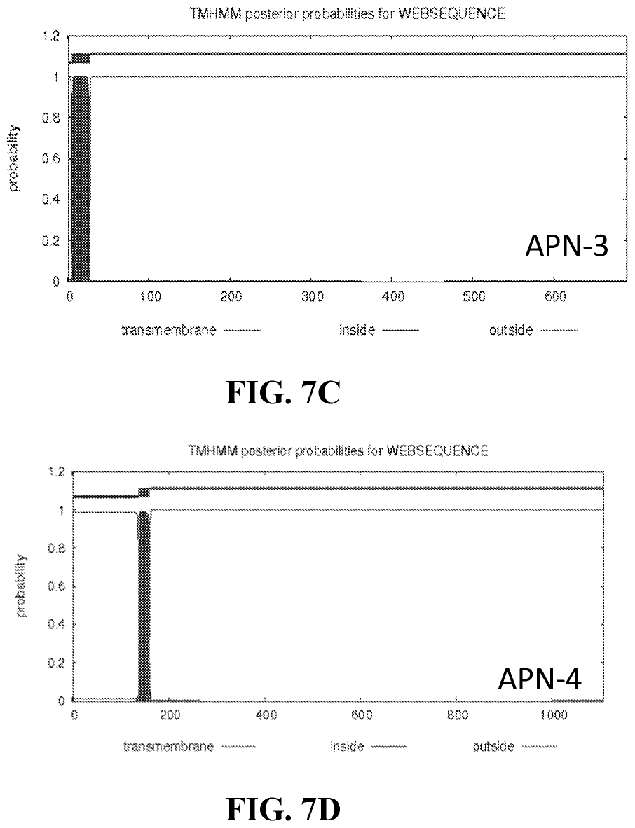

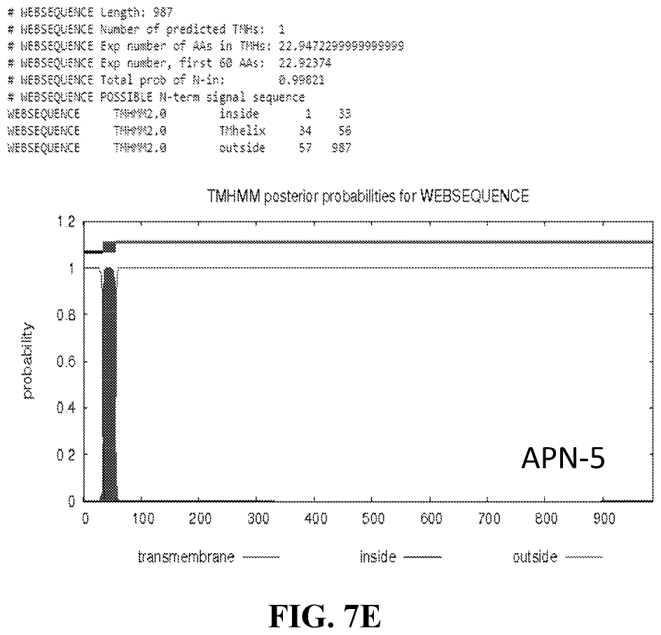

FIGS. 7A-7E show Aminopeptidase N (APN) transmembrane protein of Penaeus vannamei shrimp. The amino acid (aa) sequence of five isoforms of APN were obtained from the GenBank database (GenBank Accession No. of APN-1 is ROT67356.1; APN-2, ROT67357.1; APN-3, ROT67358.1, APN-4, ROT77087.1; APN-5, ROT72064.1). The predicted transmembrane domain was determined using TMHMM server v2.0 (http://www.cbs.dtu.dk/services/TMHMM/). The results revealed the presence of a transmembrane domain in the 5′-end of the polypeptide for each of the five APNs. APN-1 included intracellular region (aa 1 to 33), transmembrane domain (34^{th} aa-56^{th} aa), extracellular region (57^{th} aa-978^{th} aa). APN-2 includes an intracellular region (1^{st} aa-12^{th} aa), transmembrane domain (13^{th} aa-35^{th} aa), extracellular region (36^{th} aa-435^{th} aa). APN-3 included intracellular region (1^{st} aa-4^{th} aa), transmembrane domain (5^{th} aa-27^{th} aa), extracellular region (28^{th} aa-690^{th} aa). APN-4 included intracellular region (1^{st} aa-137^{th} aa), transmembrane domain (138^{th} aa-160^{th} aa), extracellular region (161^{st} aa-1107^{th} aa). APN-5 included intracellular region (1^{st} aa-33^{rd} aa), transmembrane domain (34^{th} aa-56^{th} aa), extracellular region (57^{th} aa-987^{th} aa). In each panel of APN, the blue color indicates the intracellular region, red color indicates a transmembrane domain, and pink color indicates extracellular domain. In silkworm (Bombyx mori), Aminopeptidase (APN), Alkaline phosphatase (ALP), and Cadherin (CAD) have been identified as receptors of Cry toxins. We searched GenBank database and identified homologs of APN, ALP and CAD in shrimp. Considering the similarity of structure between PirAB^{VP} and Cry toxin, we propose that APN, ALP and CAD in shrimp are receptors of PirAB^{VP} toxin. Five isoforms of APN have been identified in shrimp and each of these isoform may play different roles in binding PirAB^{VP} toxin in Vibrio parahaemolyticus pathogenesis in AHPND.

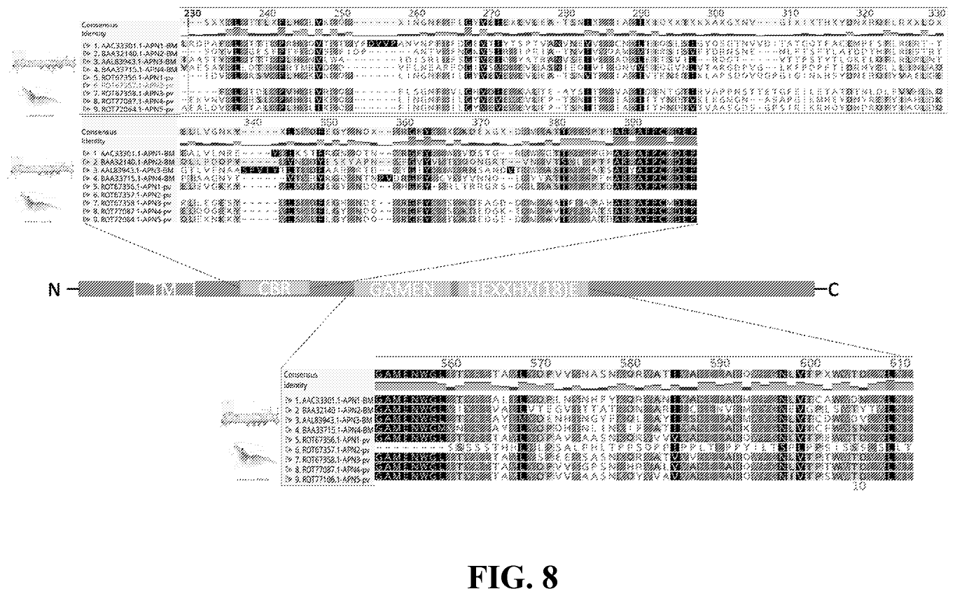

FIG. 8 shows multiple alignment of Aminopeptidase N Transmembrane protein of Penaeus vannamei shrimp with APN in silkworm Bombyx mori. The amino acid sequences of APN of silkworm and shrimp were obtained from the GenBank and aligned using a multiple alignment tool, CLUSTAL W. TM=Transmembrane, CBR=Cry Binding Region. The result shows that APNs in shrimp contain a (BR similar to APN in silkworm. The conserved residues in CBR are highlighted in black. The amino acid residues GAMEN (SEQ ID NO: 11) and HEXXHX¹⁸E (SEQ ID NO: 12) zinc-binding motifs are highly conserved in APN isoforms in shrimp. The presence of CBR and highly conserved motifs in active site of APNs in shrimp indicate that APNs in shrimp serve as receptors of PirAB^{VP} toxin.

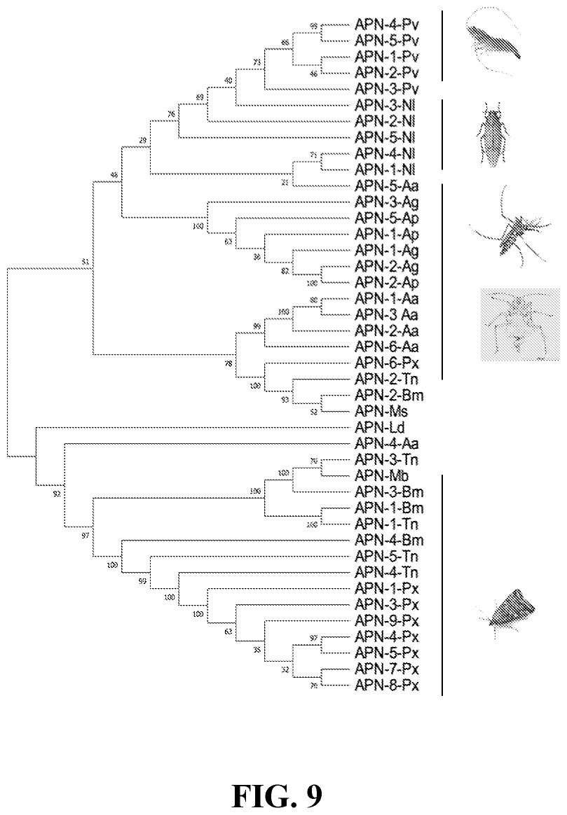

FIG. 9 shows phylogenetic tree of Aminopeptidase N Transmembrane protein of Penaeus vannamei shrimp with APN in other species. Px=Plutella xylostella; Tn=Trichoplusia ni; Bm=Bombyx mori; Mb=Mamestra brassicae; Aa=Aedes aegypti; Ld=Lymantria dispar; Ag=Aphis glycines; Ms=Manduca sexta; Ap=Acyrthosiphon pisum; NZ=Nilaparvata lugens; Pv=Penaeus vannamei. The APNs from Penaeus vannamei was a separated group suggesting APNs in shrimp may play may have different function in PirAB^{VP} binding. The phylogenetic tree was generated by using MEGA-X program with Maximum likely hood, boostrap replicates of 1000.

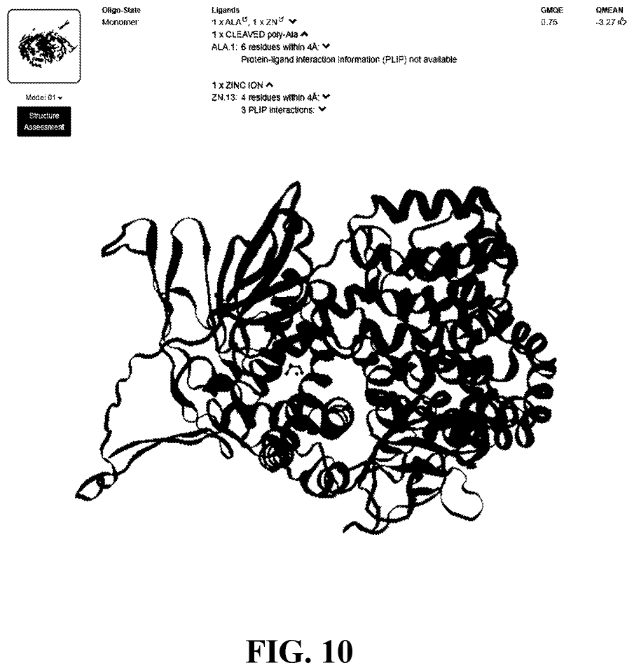

FIG. 10 shows a predicted tertiary structure of amino peptidase-N isoform 1 (APN-1) in shrimp (Penaeus vannamei). The predicted structure was generated by using a SWISS-Model (https://swissmodel.expasy.org/interactive). The APN-1 contains alpha helix and beta sheet in its tertiary structure. The GMQE and QMEAN values were 0.75 and −3.27, respectively. The model contains one Zinc ion in structure. The acceptable values of GMQE and QMEAN indicate the proposed model has a structure close to the native structure of APN-1 in vivo.

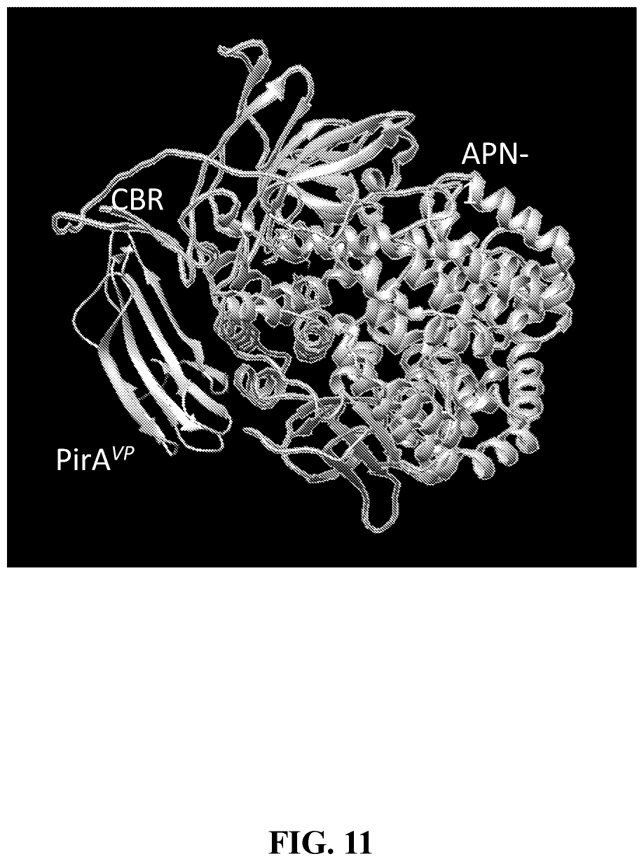

FIG. 11 shows a predicted model of interaction between PirA^{VP} and APN-1. The model was generated by using the Gramm-X server (http://vakser.compbio.ku.edu/resources/gramm/grammx/) (Tovchigrechko et al 2006). The structures of PirA^{VP} and APN-1 were built by using SWISS-model (https://swissmodel.expasy.org/interactive). The built structures of PirA^{VP} and APN-1 was submitted to Gramm-X server for modelling of interaction. Ten proposed models of interaction between PirA^{VP} and APN-1 were generated. Only one out of ten models showed the interaction between PirA^{VP} and CBR on APN-1, suggesting this model is the mode of interaction between PirA^{VP} and APN-1 in vivo in shrimp. The red-dashed circle indicated the proposed interaction region.

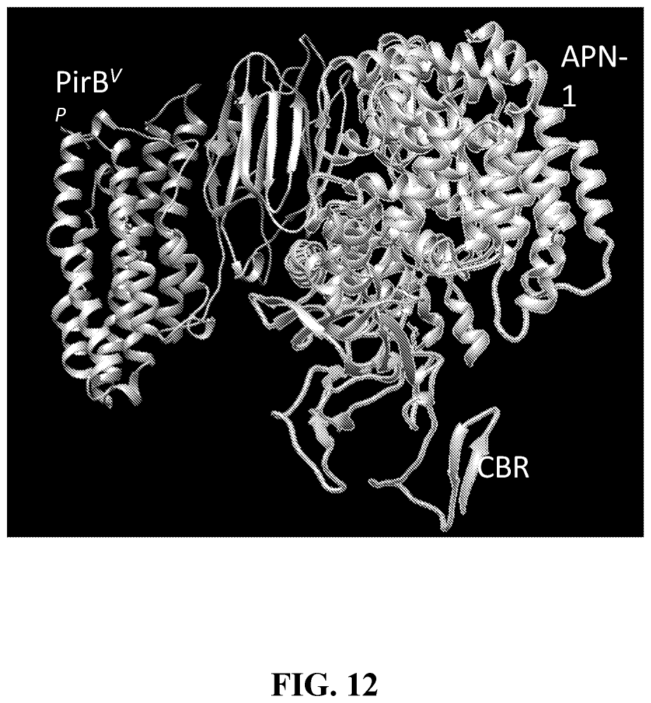

FIG. 12 shows a proposed model of interaction between PirB^{VP} and APN-1. The model was generated by using the Gramm-X server (http://vakser.compbio.ku.edu/resources/gramm/grammx/) (Tovchigrechko et al 2006). The structures of PirB^{VP} and APN-1 were built by using SWISS-model (https://swissmodel.expasy.org/interactive). The predicted structures of PirB^{VP} and APN-1 were submitted to the Gramm-X server for modelling of interaction(s). Ten proposed models of interaction between PirB^{VP} and APN-1 were generated. All ten models revealed that PirB^{VP} contacted to the helix region of the predicted tertiary structure of APN-1. None of the ten models showed any interaction between PirB^{VP} and CBR in APN-1, suggesting PirB^{VP} is not involved in the interaction of PirAB^{VP} and APN-1 in vivo.

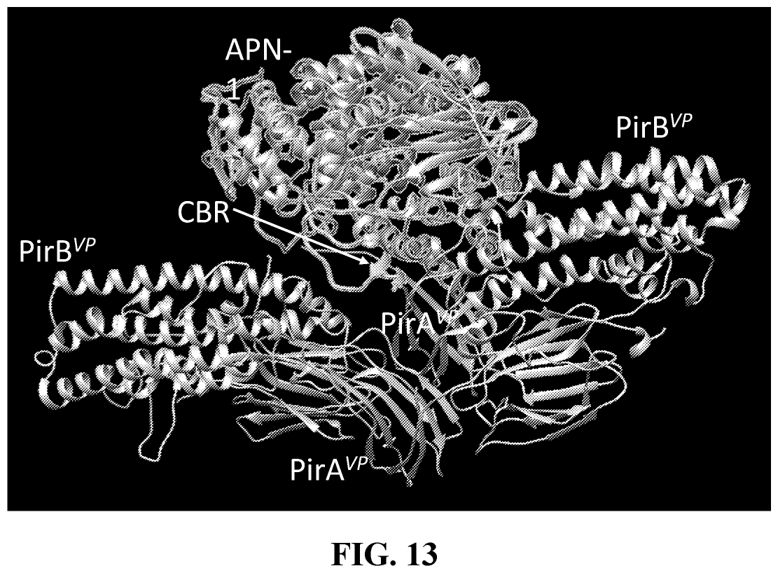

FIG. 13 shows a model of interaction between PirAB^{VP} and APN-1. The model was generated by using the Gramm-X server (http://vakser.compbio.ku.edu/resources/gramm/grammx/) (Tovchigrechko et al 2006). The structures of PirAB^{VP} and APN-1 were built by using the Gramm-X and SWISS-model (https://swissmodel.expasy.org/interactive). The predicted tertiary structures of PirAB^{VP} and APN-1 were submitted to Gramm-X server for modelling of interaction(s). Ten proposed models of interaction between PirA^{VP} and APN-1 were generated. Only one out of ten models showed the interaction between PirAB^{VP} and CBR on APN-1 via the interaction between PirA^{VP} and CBR on APN-1. This shows PirA^{VP} is involved in PirAB^{VP} and APN-1 interaction. White arrow indicated the CBR.

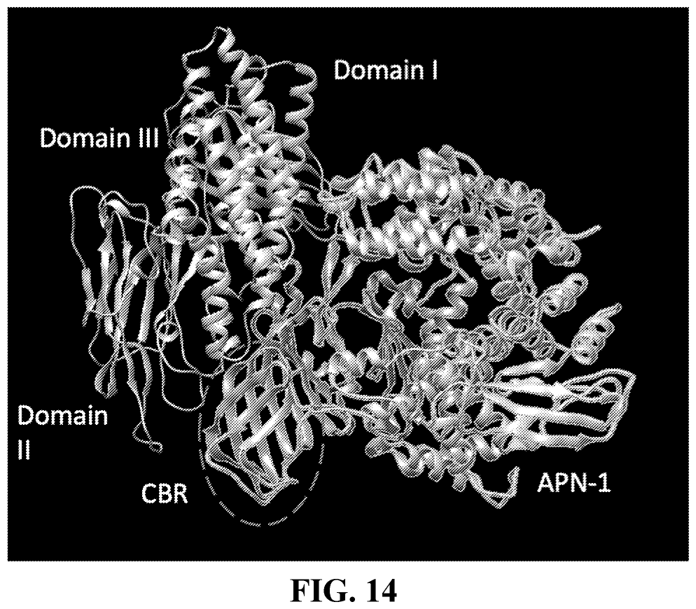

FIG. 14 shows a model of interaction between Cry1a (PDB accession number 1CIY) toxin released by Bacillus thuringiensis and APN-1 protein of Bombyx mori. The model of APN-1 from Bombyx mori was generated by SWISS-model which GMQE and QMEAN values were 0.63 and −3.78, respectively. The model of interaction was generated by using the Gramm-X server (http://vakser.compbio.ku.edu/resources/gramm/grammx/) (Tovchigrechko et al 2006). Domain II and Domain III of Cry toxin bind to the CBR (red-dashed oval) of APN-1.

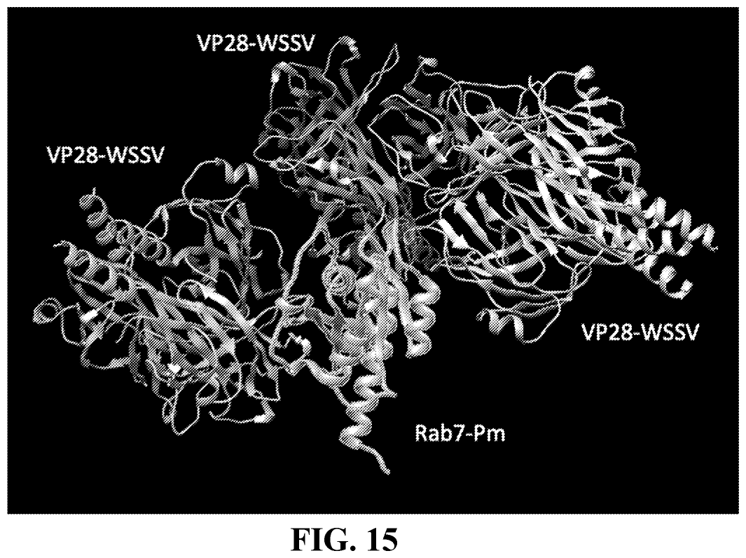

FIG. 15 shows a model of interaction between white spot virus capsid protein VP28 (PDB accession number 2ED6) and its receptor Rab7 in Penaeus monodon shrimp. The amino acid sequence of Rab7 was submitted to SWISS-model for modelling which GMQE and QMEAN values were 0.78 and −0.16, respectively. The model of interaction was generated by using the Gramm-X server (http://vakser.compbio.ku.edu/resources/gramm/grammx/) (Tovchigrechko et al 2006).

This Example can at least demonstrate that the predicted tertiary structure of PirAB^{VP} toxin produced by Vibrio parahaemolyticus causing AHPND in shrimp is similar to Cry^{Br} toxin produced by Bacillus thuringiensis in insects. This suggests the mode of action of PirAB^{VP} is likely to be similar to Cry^{Bt} toxins.

This Example can at least demonstrate that the receptors of Cry^{Bt} toxin are known that include Aminopeptidase N (APN), cadherin (CAD) and alkaline phosphatase (ALP) (Sobero'n et al., 2009; Pigott & Ellar, 2007).

This Example at least demonstrates the identification insect homologs of APN in Penaeus vannamei in the GenBank database. The predicted amino acid sequence of APNs in shrimp contain CBR domain. The CBR domain (amino acid residues 98-247) of APN is involved in binding to PirAB^{VP} toxin produced by V. parahaemolyticus.

This Example can at least demonstrate predicted tertiary structures of PirA^{VP} and PirB^{VP} show both subunits of PirAB^{VP} toxin bind to APN but only PirA^{VP} binds to the CBR domain in APN.

This Example can at least demonstrate modelling results of proposed biologically functional PirAB^{VP} toxin and APN-1 interactions that reveal only PirA^{VP} binds to APN-1.

This Example can at least demonstrate that APN serves as a receptor of PirAB^{VP} toxin produced by V. parahaemolyticus.

### Example 2—Predicted Models of Interactions Between PirA^{VP} or PirB^{VP} and APN-1, -2, -3, -4, -5

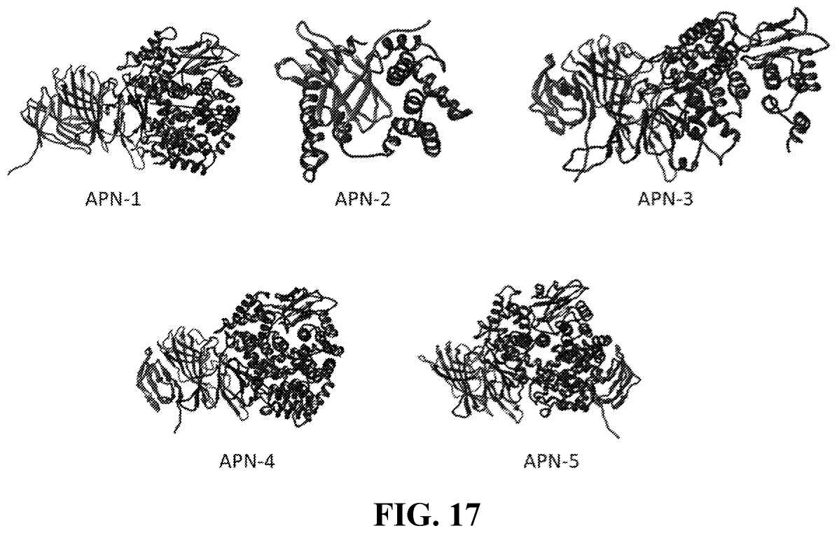

FIG. 17 shows predicted models of interaction between PirA^{VP} and APN-1, -2, -3, -4, -5. The blue indicated APN molecules. The red indicated PirA^{VP}. The green indicated Cry binding region (CBR). The interaction model were built by Haddock server (https://wenmr.science.uu.nl/).

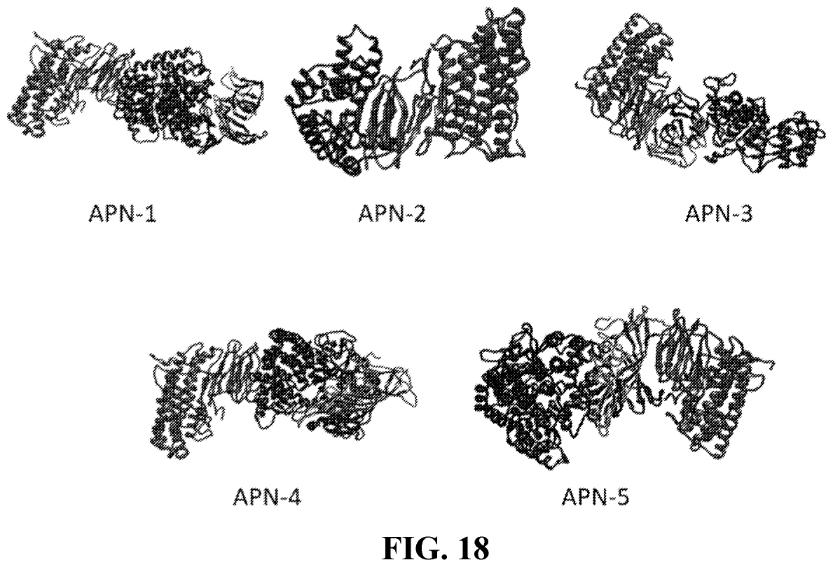

FIG. 18 shows predicted models of interaction between PirB^{VP} and APN-1, -2, -3, -4, -5. The blue indicated APN molecules. The red indicated PirB^{VP}. The green indicated Cry binding region (CBR). The interaction model were built by Haddock server (https://wenmr.science.uu.nl/).

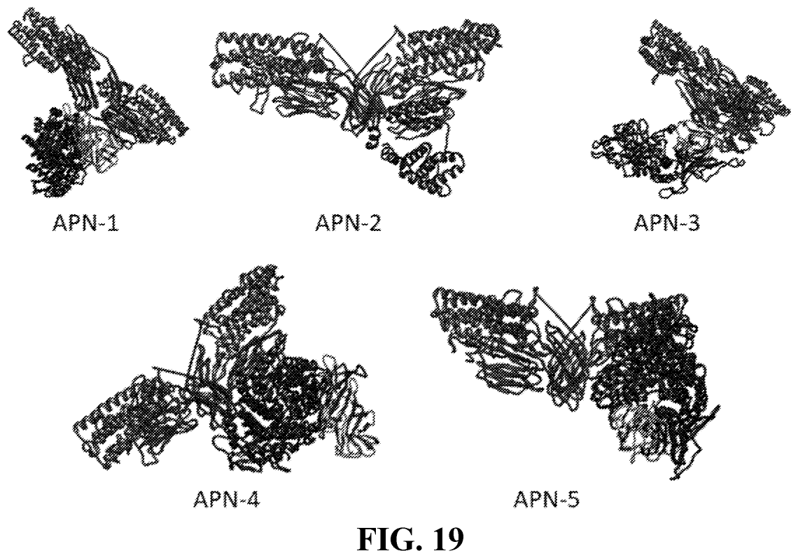

FIG. 19 shows predicted models of interaction between PirAB^{VP} and APN-1, -2, -3, -4, -5. The blue indicated APN molecules. The red indicated PirAB^{VP}. The green indicated Cry binding region (CBR). The interaction model were built by Haddock server (https://wenmr.science.uu.nl/).

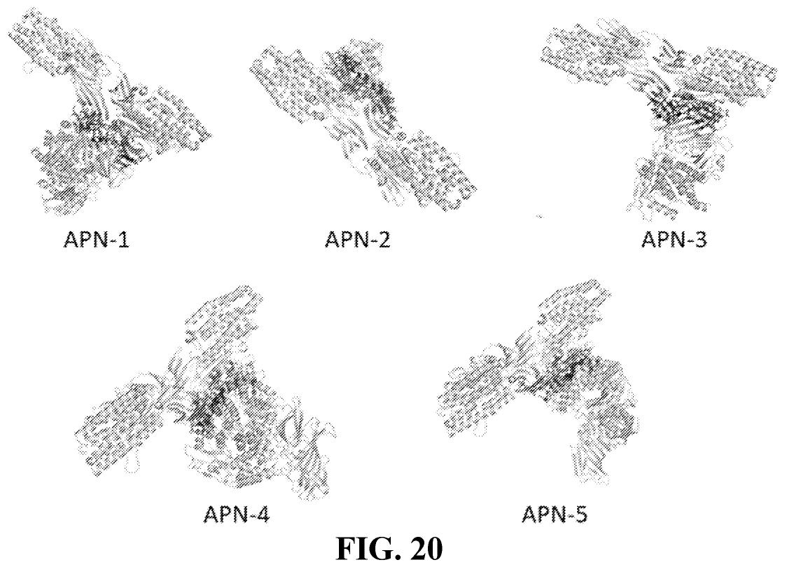

FIG. 20 shows predicted interface interaction between PirAB^{VP} and APN-1, -2, -3, -4, -5. Pink color indicated PirAB^{VP}. Light-blue color indicated APNs. Hot pink and hot blue color indicated interaction between PirAB^{VP} and APNs. The interaction interface were built by Prodigy server (https://wenmr.science.uu.nl/prodigy/).

Table 1 shows a summary of prediction of binding of Vibrio parahaemolyticus PirA^{VP}, PirB^{VP}, PirAB^{VP} toxin to shrimp aminopeptidase N (APN) isoforms 1 to 5. The work was done by using Prodigy server (https://wenmr.science.uu.nl/prodigy/). The lower values indicated the stronger binding force.

TABLE 1

| | Binding affinity (ΔG)(kcal/mol) | | |
|---|---|---|---|
| APNs | PirA^{VP} | PirB^{VP} | PirAB^{VP} |
| APN-1 | −14.9 | −11.5 | −15.3 |
| APN-2 | −15.9 | −15.2 | −15.4 |
| APN-3 | −15.5 | −17.8 | −17.5 |
| APN-4 | −12.9 | −15.8 | −17.9 |
| APN-5 | −16.3 | −15.7 | −14.5 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A Vibrio spp. toxin binding protein, wherein the Vibrio spp. toxin binding protein is capable of binding or otherwise interacting with a Vibrio spp. toxin.
2. The Vibrio spp. toxin binding protein of aspect 1, wherein the Vibrio spp. toxin is PirA, PirB, a PirA-like toxin, a PirB-like toxin, or a combination thereof.
3. The Vibrio spp. toxin binding protein of any one of the preceding aspects, wherein the Vibrio spp. toxin binding protein is aminopeptidase-N or a fragment thereof, alkaline phosphatase (ALP) or a fragment thereof, midgut membrane-bound cadherin (CAD) or a fragment thereof, or a combination thereof.
4. The Vibrio spp. toxin binding protein of any one of the preceding aspects, wherein the Vibrio spp. toxin binding protein comprises a cry protein binding region (CBR), a GAMEN (SEQ ID NO: 11) motif, a Zn²⁺ binding region, or a combination thereof.
5. The Vibrio spp. toxin binding protein of any one of the preceding aspects, wherein the Vibrio spp. toxin binding protein is a recombinant protein.
6. The Vibrio spp. toxin binding protein of any one of the preceding aspects, wherein the Vibrio spp. toxin binding protein is modified to have increased binding affinity and/or specificity for one or more Vibrio spp. toxins.
7. The Vibrio spp. toxin binding protein of any one of the preceding aspects, wherein the Vibrio spp. toxin binding protein comprises a polypeptide that is 50-100% identical to any one of SEQ ID NO: 2, 4, 6, 8, or 10 or is a fragment thereof of at least 3 amino acids.
8. A formulation comprising a Vibrio spp. toxin binding protein or a fragment thereof of any one of the preceding aspects; and a suitable carrier.
9. The formulation of aspect 8, wherein the formulation is a feed formulation suitable for a crustacean.
10. The formulation of aspect 9, wherein the crustacean is a shrimp.
11. The formulation of any one of the preceding aspects, wherein the formulation is adapted for delivery in a water source.
12. The formulation of any one of the preceding aspects, wherein the formulation is effective to treat or prevent an infection, disease, or a symptom thereof of a Vibrio spp. organism in a shrimp.
13. The formulation of aspect 12, wherein the disease is AHPND.
14. A polynucleotide that encodes any one of the Vibrio spp. toxin binding protein or a fragment thereof of any one of aspects 1-7.
15. A vector or vector system comprising a polynucleotide of aspect 14.
16. A cell comprising a polynucleotide of aspect 9 or a vector or vector system of aspect 15.
17. The cell of aspect 16, wherein the cell is capable of producing the Vibrio spp. toxin binding protein.
18. A method of treating or preventing a Vibrio spp. infection or disease comprising: administering an amount of a protein, vector or vector system, cell, or formulation as in any one of the preceding aspects to a subject.
19. The method of aspect 18, wherein the subject is a non-human animal.
20. The method of aspect 18 or 19, wherein the subject is a crustacean.
21. The method of any one of the preceding aspects, wherein the subject is a shrimp.
22. The method of any one of the preceding aspects, wherein administrating is via a feed or water source.
23. The method of any one of the preceding aspects, wherein the is infected with, is suspected of being infected with an organism of a Vibrio spp., or will be exposed to an organism of a Vibrio spp.
24. A method of detecting an acute hepatopancreatic necrosis disease (AHPND) and/or AHPND susceptible organisms and/or cells therefrom comprising:
   detecting in one or more cells an AHPND susceptibility signature, wherein the AHPND tolerance signature comprises:
      i. one or more genes selected from APN, ALP, or CAD; and/or
      ii. one or more SNPs in one or more genes selected from APN, ALP, or CAD; and
   wherein detecting of the susceptibility signature indicates that the organism and/or cell is tolerant/resistant or susceptible to AHPND.
25. The method of aspect 24, wherein the organism is a crustacean.
26. The method of any one of aspects 24-25, wherein the organism is a crab, lobster, crayfish, shrimp, prawn, or krill.
27. The method of any one of aspects 24-26, wherein the organism is obtained from a cell, an organ, a tissue, a bodily fluid, or a combination thereof.
28. The method of any one of aspects 24-27, wherein the sample is obtained from a hepatopancreas, gut, gill, hemocytes, or a combination thereof of the organism or a representative contemporary.
29. A method of treating or preventing a Vibrio spp. infection or disease comprising:
   a. detecting an AHPND susceptible organism by performing a method as in any one or more of aspects 24-27; and
   b. performing a method as in any one of aspects 18-23.

What is claimed is:

1. A Vibrio spp. toxin binding protein, wherein the Vibrio spp. toxin binding protein is capable of binding or otherwise interacting with a Vibrio spp. toxin.

2. The Vibrio spp. toxin binding protein of claim 1, wherein the Vibrio spp. toxin is PirA, PirB, a PirA-like toxin, a PirB-like toxin, or a combination thereof.

3. The Vibrio spp. toxin binding protein of claim 1, wherein the Vibrio spp. toxin binding protein is aminopeptidase-N or a fragment thereof, alkaline phosphatase (ALP) or a fragment thereof, midgut membrane-bound cadherin (CAD) or a fragment thereof, or a combination thereof.

4. The Vibrio spp. toxin binding protein of claim 1, wherein the Vibrio spp. toxin binding protein comprises a cry protein binding region (CBR), a GAMEN (SEQ ID NO: 11) motif, a Zn²⁺ binding region, or a combination thereof.

5. The Vibrio spp. toxin binding protein of claim 1, wherein the Vibrio spp. toxin binding protein is a recombinant protein.

6. The Vibrio spp. toxin binding protein of claim 1, wherein the Vibrio spp. toxin binding protein is modified to have increased binding affinity and/or specificity for one or more Vibrio spp. toxins.

7. The Vibrio spp. toxin binding protein of claim 1, wherein the Vibrio spp. toxin binding protein comprises a polypeptide that is 50-100% identical to any one of SEQ ID NO: 2, 4, 6, 8, or 10 or is a fragment thereof of at least 3 amino acids.

8. A formulation comprising:
a Vibrio spp. toxin binding protein or a fragment thereof of claim 1; and
a suitable carrier.

9. The formulation of claim 8, wherein the formulation is a feed formulation suitable for a crustacean.

10. The formulation of claim 9, wherein the crustacean is a shrimp.

11. The formulation of claim 8, wherein the formulation is adapted for delivery in a water source.

12. The formulation of claim 8, wherein the formulation is effective to treat or prevent an infection, disease, or a symptom thereof of a Vibrio spp. organism in a shrimp.

13. The formulation of claim 12, wherein the disease is AHPND.

14. A polynucleotide that encodes any one of the Vibrio spp. toxin binding protein or a fragment thereof of any one of claims 1-7.

15. A vector or vector system comprising: a polynucleotide of claim 14.

16. A cell comprising: a polynucleotide of claim 9 or a vector or vector system of claim 15.

17. The cell of claim 16, wherein the cell is capable of producing the Vibrio spp. toxin binding protein.

18. A method of treating or preventing a Vibrio spp. infection or disease comprising:
administering an amount of a protein of claim 1, a vector or vector system capable of expressing the protein of claim 1, cell comprising and/or expressing a protein of claim 1 and/or a vector or vector system capable of expressing the protein of claim 1, or a formulation comprising the protein of claim 1, the vector or vector system, the cell to a subject.

19. The method of claim 18, wherein the subject is a non-human animal.

20. The method of claim 19, wherein the subject is a crustacean.

21. The method of claim 20, wherein the subject is a shrimp.

22. The method of claim 18, wherein administrating is via a feed or water source.

23. The method of claim 18, wherein the subject is infected with, is suspected of being infected with an organism of a Vibrio spp., or will be exposed to an organism of a Vibrio spp.

24. A method of detecting an acute hepatopancreatic necrosis disease (AHPND) and/or AHPND susceptible organisms and/or cells therefrom comprising:
detecting in one or more cells an AHPND susceptibility signature, wherein the AHPND tolerance signature comprises:
i. one or more genes selected from APN, ALP, or CAD;
ii. one or more SNPs in one or more genes selected from APN, ALP, or CAD;
wherein detecting of the susceptibility signature indicates that the organism and/or cell is tolerant/resistant or susceptible to AHPND.

25. The method of claim 24, wherein the organism is a crustacean.

26. The method of claim 24, wherein the organism is a crab, lobster, crayfish, shrimp, prawn, or krill.

27. The method of claim 24, wherein the organism is obtained from a cell, an organ, a tissue, a bodily fluid, or a combination thereof.

28. The method of claim 24, wherein the sample is obtained from a hepatopancreas, gut, gill, hemocytes, or a combination thereof of the organism or a representative contemporary.

29. A method of treating or preventing a Vibrio spp. infection or disease comprising:
detecting an AHPND susceptible organism by performing a method as in claim 24.

30. A method of treating or preventing a Vibrio spp. infection or disease comprising:
performing a method as in claim 18.
